(19) United States
(12) Patent Application Publication
(10) Pub. No.: US 2012/0283306 A1
(43) Pub. Date: Nov. 8, 2012

(54) MOUSE MODEL FOR AVM

(75) Inventors: Tsugio Seki, Martinez, GA (US); Cargill Alleyne, JR., Martinez, GA (US); Nathan Yanasak, Crawfordville, GA (US)

(73) Assignee: Georgia Health Sciences University, Augusta, GA (US)

(21) Appl. No.: 13/456,935

(22) Filed: Apr. 26, 2012

Related U.S. Application Data
(60) Provisional application No. 61/479,832, filed on Apr. 27, 2011.

(51) Int. Cl. A01K 67/027; G01N 33/48; C40B 30/04; G01N 33/53; A61K 31/415; C12Q 1/68

(52) U.S. Cl. 514/406; 800/9; 435/6.12; 506/9; 436/510; 435/7.1; 800/3

(57) ABSTRACT

Arteriovenous malformation, or Arteriovenous vascular malformation (AVM) is a congenic disorder characterized by an abnormal connection between veins and arteries, resulting in hemorrhaging and even death. A lack of good animal models has long been an obstacle for identifying effective drugs for neurological AVM treatment. Describe herein is a mouse model for AVM that includes a viable, postnatal animal with a conditional deletion of the activin receptor-like kinase 1 (Alk1;Acvrl1). The Alk1-cKO mouse model can be used to identify genes and gene products that are upregulated in subjects suffering from AVM. For example, it has been discovered Agpt2, IL1β, and TNF-α, are upregulated in Alk1-cKO compared to controls. Pharmaceutical compositions for treatment of AVM are disclosed. Preferred compositions inhibit or decrease expression of angiogenic and pro-inflammatory factors, such as VEFG, Cox-2, Agpt2, IL1β, TNF-α, and matrix metalloproteinases. Methods of determining efficacy of potential AVM therapeutics are also disclosed.

### CROSS REFERENCE TO RELATED APPLICATIONS

This application claims priority to and benefit of U.S. Provisional Application No. 61/479,832 filed Apr. 27, 2011, and where permissible is incorporated herein in its entirety.

### FIELD OF THE INVENTION

The present application is generally related to compositions and methods for preventing hemorrhages from arteriovenous vascular malformations, and a disease model for testing their efficacy.

### BACKGROUND OF THE INVENTION

Arteriovenous malformation, or Arteriovenous Vascular Malformation (AVM) is a vascular disorder characterized by an abnormal connection between veins and arteries. Although AVM typically occurs between veins and arteries of the central nervous system, it can occur between veins and arteries anywhere in the body. In a normal functioning human body, arteries carry oxygen-rich blood away from the heart to the rest of the body, and veins return oxygen-depleted blood to the lungs and heart. AVM interferes with this cyclical process. Instead of the gradual transition through the capillaries from arteries to veins that is typical of normal functioning vasculature, AVMs cause direct associations between arteries and veins.

Roughly 88% of people affected with AVM are asymptomatic; often the malformation is discovered as part of an autopsy or during treatment of an unrelated disorder (referred to as “an incidental finding”). In rare cases, the expansion or a micro-bleed from an AVM in the brain can cause epilepsy, oxygen deficit or pain. General symptoms of a cerebral AVM include headache and epilepsy. Other symptoms depend on the location of the malformation and include: difficulties with movement or coordination, including muscle weakness and even paralysis; vertigo (dizziness); difficulties of speech (dysarthria) and communication, such as aphasia; difficulties with everyday activities, such as apraxia; abnormal sensations (numbness, tingling, or spontaneous pain); hallucinations. In humans, bleeding from AVM can cause severe neurological disability or death.

There is currently no effective treatment option for some of the patients who have complex AVMs. Although various surgical, endovascular, and radiosurgical treatment options are available for neurological AVMs, patients with severe AVMs are often diagnosed as untreatable and forced to live with the possibility of sudden, potentially life-ending hemorrhages. In addition, treatable patients who are selected for radiosurgery also continue to bear a risk of hemorrhage during the course of treatment for a few years. Furthermore, patients with minor AVMs that have not ruptured face difficulties determining whether the future risk of hemorrhage outweighs the risks of prophylactic interventions. Currently the mechanisms leading a silent AVM to a life-threatening hemorrhage are undefined. Thus, although prevention of hemorrhage from AVMs is critical and vital, a lack of understanding of the mechanisms has been a barrier to the discovery of drugs that lowers the chance of hemorrhage.

Therefore, it is an object of the invention to identify molecular targets for reducing or preventing AVM formation or hemorrhages from AVM lesions.

It is another object of the invention to provide compositions and methods for reducing or preventing AVM formation or complications related to AVMs.

It is still another object of the invention to provide methods for determining the effectiveness and efficacy of compositions and methods for reducing or preventing AVM formation or complications arising from AVM lesions.

### SUMMARY OF THE INVENTION

A transgenic non-human animal model of Arteriovenous Vascular Malformation (AVM) is provided. One embodiment provides a transgenic mouse model for AVM having a conditional deletion of activin receptor-like kinase 1 (Alk1;Acvrl1). The disclosed transgenic mouse (referred to as an Alk1-cKO mouse) is useful for developing and identifying new therapeutic agents for the treatment or prevention of AVM. For example, the Alk1-cKO mouse model is useful for identifying biomarkers including genes and gene products that are therapeutic targets for the treatment or prevention of AVM. Representative biomarkers associated with AVM include but are not limited to Agpt2, IL1β, and TNF-α.

Pharmaceutical compositions and methods for treatment of AVM are disclosed. Preferred compositions inhibit or decrease expression of angiogenic and pro-inflammatory factors, such as VEGF, Cox-2, Agpt2, IL1β, TNF-α, and matrix metalloproteinases. AVM can be treated with a Cox-2 inhibitor, such as Celecoxib, or a matrix metalloproteinase inhibitor, such as doxycycline.

Disclosed are methods of reducing or preventing hemorrhaging from AVM lesions that involve administering an effective amount of a therapeutic composition. The therapeutic composition can reduce or inhibit an angiogenic or inflammatory factor selected from the group consisting of VEGF, Agpt2, Il-1β, TNF-α, Cox-2, and matrix metalloproteinase.

Also disclosed are methods for identifying molecular targets for therapeutic treatment of AVM. The methods involve identifying genes or proteins differentially expressed in a biological sample from a conditional knockout mouse compared to a control. The differential expression of genes can be measured using quantitative polymerase chain reaction, microarray analysis, or northern blot analysis and the differential expression of proteins can be measured using immunohistochemistry, western blot, mass spectroscopy, spectrophotometry, enzyme immunoassay, or a radioimmunoassay.

Methods of determining efficacy of potential AVM therapeutics are also disclosed. The methods can involve administering a candidate therapeutic to a first conditional knockout mouse and determining if the therapeutic prevents or reduces symptoms of AVM compared to a second conditional knockout mouse that is not administered the therapeutic. The symptoms of AVM can be the number or severity of AVM-associated hemorrhages or can be an increased expression of angiogenic or inflammatory factors. The angiogenic or inflammatory factor can be selected from the group consisting of VEGF, Agpt2, Il-1β, TNF-α, Cox-2, or a matrix metalloproteinase.

### BRIEF DESCRIPTION OF THE DRAWINGS

FIGS. 1A and 1B are schematic diagrams. FIG. 1A shows the cell type-specific Alk1 deletion using Cre-loxP system. FIG. 1B shows what happens in an Alk1 deletion with and without insult.

FIG. 2 is an Alk1-cKO survival curve showing survival versus age (weeks). The endpoints were death and paralysis. The lethal period (LP) and stable period (SP) are marked with dashed arrows.

FIGS. 3A and 3B show graphs of AVM score in brain versus age (weeks). AVMs with (a) and without (b) hemosiderin deposition were independently scored.

FIG. 4 is a bar graph showing fold difference versus Agpt2, Il-1β and TNF-α using quantitative PCR analysis of brain tissues (n=6 each).

FIG. 5 is a flow chart of the mating scheme used to produce the Alk1-cKO mice.

FIG. 6 shows the experimental timeline used to determine the AVM grading of Alk1-cKO mice.

FIG. 7 shows the experimental timeline used to investigate the histology of the Alk1-cKO mice.

FIG. 8 shows a flow chart of the mating scheme used to generate the experiment genotype.

FIG. 9 shows the experimental timeline used to study AVMs/TVs in the skin using the blue latex injection.

FIG. 10 shows the experimental timeline used to study AVM/TV formation using histological analyses.

FIG. 11 shows the experimental design of studying the effects of Celecoxib.

FIG. 12 is a line graph of Alk1-cKO survival curves showing survival versus age (weeks).

FIG. 13 shows the experimental design for examining the molecular and histological effects of Celecoxib.

FIG. 14 shows the experimental design for determining mortality rat in mice on Doxycycline.

FIG. 15 shows the experimental design for examining different molecular and histological effects of Doxycycline.

FIG. 16 provides the survival curves of SM22Cre-del models showing survival versus age (weeks). End points were hindlimb paralysis, whole body paralysis, and spontaneous death. Note that plots for control genotypes overlap at the top. The numbers of SM22Cre-del mice at risk (Number at Risk; NR) are indicated in the parentheses on the X axis.

FIG. 17 shows the AVM scoring system for humans and mice,

FIG. 18 shows line graphs of AVM scores vs. age (weeks) of mice. Plots of AVM score in stable period (SP) mice against age. (two left hand graphs) Brain AVM/TV/+H (+hemosiderin) and AVM/TV/−H (no hemosiderin) scores of 15 SM22Cre-del mice (solid circle) and 22 littermate controls (open circle) were determined using the mouse AVM grading system outlined in Table 1 and are plotted against age. Solid and dashed lines indicate trend lines for SM22Cre-del and control mice, respectively. Note that all 15 SM22Cre-del mice had multiple AVMs/TVs in the brain and showed consistently high scores for both AVM/TV/+H and AVM/TV/−H throughout SP and no trends of increase, suggesting little or no increase in severity of AVM during SP. The brain AVM/TV/−H score in 22 control littermates showed positive values due to the cerebellar TVs. (two right hand graphs) Spinal cord AVM/TV/+H and AVM/TV/−H scores of the same set of animals were determined using the same grading system and are plotted. Note that spinal AVMs/TVs were found in 10 animals however, most of these AVMs/TVs were not accompanied by hemosiderin deposition. This may be due to prior removals of paralyzed animals during lethal period (LP) since spinal hemorrhage generally caused hind limb paralysis.

FIG. 19 shows bar graphs of fold difference versus 31 different candidate genes. The graphs represent the quantitative PCR (qPCR) analysis of SM22Cre-del mouse brain tissues (n=6 per group). Two statistical analysis results are shown. The first is ANOVA assay using all four groups and the results are shown in black brackets and asterisks (** for p<0.01, * or p<0.05). The second is Student t-test using the two LP groups and the results are shown in red brackets and asterisks. Note that the Taqman Assay for Alk1 gene amplifies and detects the transcript at exons 10-11 region, whereas the Alk1-flox recombination/deletion occurs at exons 4-6. Therefore, the Alk1 assay is collectively detecting both recombined and nonrecombined transcripts. Hence, the increase in total Alk1 transcript may be caused by compensatory upregulation of Alk1-null transcript evoked by ALK1 protein deficiency. Endoglin (Eng) upregulation may also be induced by the ALK1 protein absence since these two molecules are both involved in HHT pathogenesis in human and belong to the same TGF-β superfamily signaling pathways.

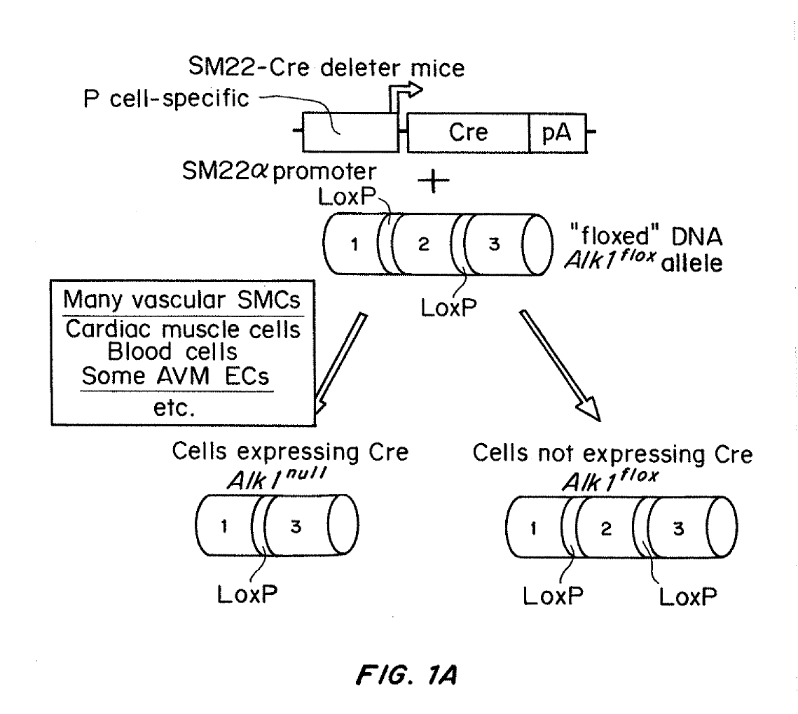

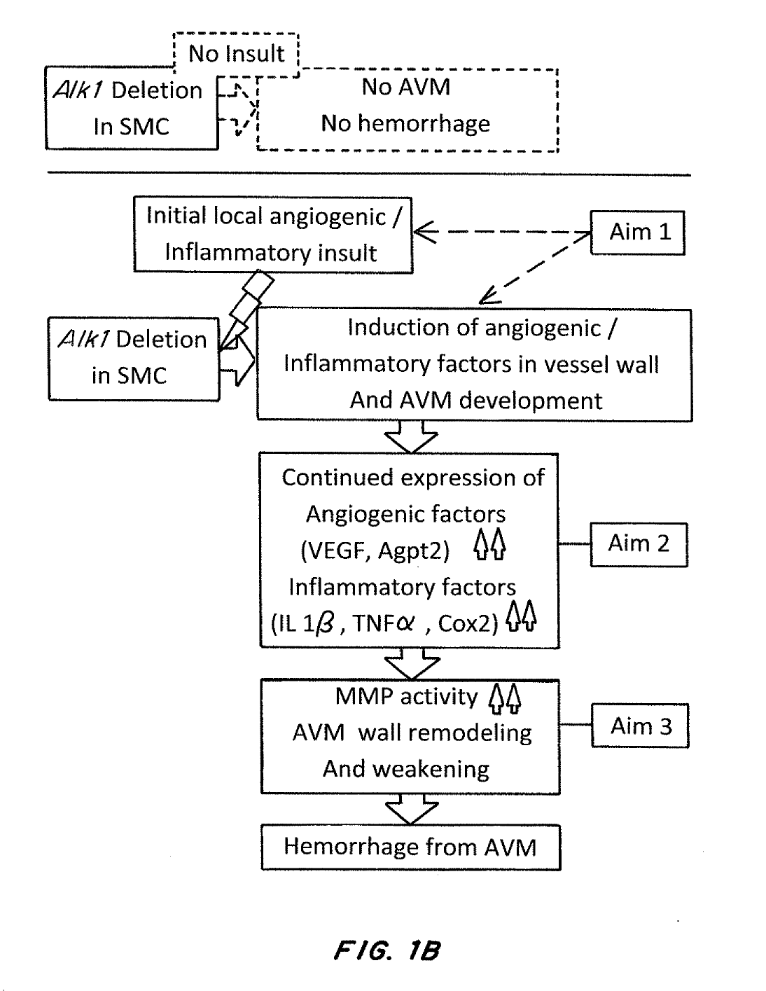

### DETAILED DESCRIPTION OF THE INVENTION

### I. Definitions

“AVM” refers to Arteriovenous Vascular Malformation.

The term “effective amount” refers to an amount of peptide or conjugate sufficient for binding to the target protein or interfering with reactive oxygen and nitrogen species modifications. The exact amount required will vary from subject to subject, depending on the age, and general condition of the subject, the organ or tissue that is being targeted, the particular peptides or conjugates used, and its mode of administration. An appropriate “effective amount” can be determined by one of ordinary skill in the art using only routine experimentation.

As used herein, the term “subject” means any individual who is the target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “treat,” “treating,” and “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### II. Mouse Model for Arteriovenous Vascular Malformation

A lack of good animal models has long been an obstacle for identifying effective drugs for neurological AVM treatment. Mouse models of AVM have been generated mainly by genetic modification of two genes, Alk1 and endoglin (Eng). In humans, mutations in these genes cause hereditary hemorrhagic telangiectasia (HHT), which is characterized by AVMs in various organs. Conventional Alk1 and Eng knockout mice are deficient as disease models because they are embryonic lethal. Inducible knockout technology results in 100% lethality in less than three weeks due to severe vascular malformation and hemorrhage in the lungs. The too negligible or too drastic AVM phenotypes in the previous models have made them unsuitable for assessing drug treatment effects.

A preferred animal model for AVM is a viable, postnatal animal with a conditional deletion of activin receptor-like kinase 1 (Alk1;Acvrl1). Preferably, they survive in spite of carrying AVMs. Preferably, the animals survive at least 2 weeks, more preferably at least 5 weeks, most preferably at least about 10 weeks. In the most preferred embodiment, the animals produce/harbor multiple AVMs that can become as large as a quarter of brain hemisphere and can bleed.

Transgenic Animals

The AVM mouse model disclosed herein is typically characterized by conditional knockout (cKO) of the activin receptor-like kinase 1 (Alk1;Acvrl1), referred to herein as (Alk1-cKO).

In one embodiment Alk1-cKO mice are generated by intercrossing Alk1(+/−) heterozygous mice, Alk1(flox/flox) conditional deletion mice, and Tg(SM22-Cre) mice (Jackson Laboratory stock #004746). Alk1(+/−) heterozygous mice are describe in Seki, et al., Circ Res. 93:682-9 (2003), Alk1(flox/flox) conditional deletion mice are described in Park, et al., Blood, 111:633-42 (2008), and Tg(SM22-Cre) mice are described in Holtwick, Proc Natl Acad Sci USA. 99:7142-7 (2002), each of which is incorporated by reference in its entirety. The parental mice can be purchased from commercial vendors such as the Jackson Laboratory or prepared according to methods of making transgenic or knockout mice that are well known in the art, see for example Transgenic Mouse: Methods and Protocols (Hofker and Deursen, ed.) Humana Press Inc., New Jersey (2003) and Manipulating the Mouse Embryo, John Inglis, New York (2003) both of which are incorporated by reference in their entirety.

Alk1(flox/flox) mice have been utilized previously with other Cre-deletion mouse lines and showed 100% lethal phenotype due to severe internal hemorrhages. Both cKO genotypes, Alk1(flox/flox);Tg(SM22-Cre) and Alk1(flox/−);Tg(SM22-Cre), lack functional Alk1 gene in the smooth muscle cells (SMCs) due to early embryonic Cre recombination by Tg(SM22-Cre) transgene. The mice exhibit hemorrhages from AVMs in the central nervous system, which causes paralysis and lethality.

In some embodiments, the Alk1-cKO is further genetically modified to express VEGF.

### III. Methods of Identifying Molecular Targets for Pharmaceutical Therapy

As discussed in the Examples below, it has been discovered that angiogenic and inflammatory factors are increased in a mouse model of AVM. The Examples below show that factors including, but not limited to, Agpt2, IL1β, and TNF-α, are upregulated in Alk1-cKO mice compared to controls. Therefore, the Alk1-cKO mouse model can be used to identify additional genes and gene products that are upregulated in subjects suffering from AVM.

For example, differential expression of genes or proteins in a subject suffering from AVM can be identified by determining the level of an mRNA or protein in a biological sample from the Alk1-cKO and comparing it to a control sample, for example an equivalent biological sample from a wildtype mouse. Expression of genes (i.e. quantification of mRNA level) can be measured using any method known in the art, for example quantitative polymerase chain reaction, microarray analysis, or northern blot analysis. Expression of a gene product (i.e. a protein) can be measured using any method known in the art for example, immunohistochemistry, western blot, mass spectroscopy, spectrophotometry, enzyme immunoassay, or a radioimmunoassay. Genes or gene products that are differentially expressed in Alk1-cKO compared to a control are targets for therapeutic treatment of AVM. For example if gene expression or gene product expression is decreased in the Alk1-cKO, the therapeutic composition may increase gene expression or gene product expression. If gene expression or gene product expression is increased in the Alk1-cKO, the therapeutic composition may decrease gene expression or gene product expression.

### IV. Methods of Treating AVM

Methods for treating one or more symptoms of AVM are provided. For example, factors that are upregulated or downregulated in AVM, such as Agpt2, IL1β, and TNF-α, can be targeted.

Representative methods of treatment include administering to the subject an effective amount of an inhibitor of angiogenic or pro-inflammatory factors. Suitable inhibitors include celecoxib, doxycycline, adalimumab, golimumab infliximab, natalizumab, and etanercept.

A. Combination Therapy

In certain embodiments, the methods for treating AVM include administering additional therapeutic agents that may or may not inhibit or reduce the identified targets, such as Agpt2, IL1β, and TNF-α. For example, the disclosed therapeutic agents can be administered in combination with known AVM treatments and treatments for AVM symptoms, such as, but not limited to, headache, back pain, seizures, vertigo, dementia, and hallucinations. Examples of therapeutic agents are anti-inflammatories and stereotactic radiosurgery.

For combination therapies, the therapeutic treatment can be co-administered with another therapeutic agent. Co-administration involves the administration of two or more agents to a subject so that both agents and/or their metabolites are present in the animal at the same time. Co-administration includes simultaneous administration in separate compositions, administration at different times in separate compositions, or administration in a composition in which both agents are present.

In one embodiment, a therapeutic agent can be administered in the period between the time of AVM discovery and the surgical removal/endovascular embolization of AVM. In this instance, the combination therapy is a combination of therapeutic agent and surgery. This type of treatment can stabilize the vascular wall and make it stronger, lowering the chances of hemorrhage during the surgical procedures.

B. Administration

The disclosed compositions can be administered in any suitable manner. The manner of administration can be chosen based on, for example, whether local or systemic treatment is desired, on the area to be treated, and on what type of composition is being delivered (e.g., peptide, nucleic acid, etc.). For example, the compositions can be administered orally, parenterally (e.g., intravenous, subcutaneous, or intramuscular injection), or the like.

For oral administration, solid dosage forms of tablets, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The peptides can also be in micro-encapsulated form, if appropriate, with one or more excipients.

Peptides may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. For example, PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or derivative) or by release of the peptide (or derivative) beyond the stomach environment, such as in the intestine.

To ensure full gastric resistance a coating can be impermeable to at least pH 5.0. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

To aid dissolution of peptides into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of peptides are for instance the fatty acids oleic acid, linoleic acid and linolenic acid.

Controlled release oral formulations may be desirable. The peptides could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The peptides could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

Parenteral administration of the composition is generally characterized by injection. Injectables can be prepared in conventional forms, either as liquid solutions or suspensions, solid forms suitable for solution of suspension in liquid prior to injection, or as emulsions. Parenteral administration can involve the use of a slow release or sustained release system such that a constant dosage is maintained.

The compositions can be delivered via a catheter or similar device that allows for direct administration of the composition to the area of interest, such as the brain.

C. Additional Ingredients

The compositions disclosed herein can be administered to a subject along with additional ingredients. As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Additional ingredients can also include a targeting agent wherein the therapeutic agent is targeted to specific cells or tissue in the body. For example, the therapeutic agent can be targeted to the arteries. Therapeutic agents can be encapsulated within, dispersed in, associated with, or conjugated to a nanoparticle functionalized with one or more targeting agents.

The nanoparticles may be folioed from one or more polymers, copolymers, or polymer blends. In some embodiments, the one or more polymers, copolymers, or polymer blends are biodegradable. Examples of suitable polymers include, but are not limited to, polyhydroxyacids such as poly(lactic acid), poly(glycolic acid), and poly(lactic acid-co-glycolic acids); polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals ; polycyanoacrylates; polyacrylates; polymethylmethacrylates; polysiloxanes; poly(oxyethylene)/poly(oxypropylene)copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting. In some cases, the mitochondrial targeting agents are polypeptides that are covalently linked to the surface of the nanoparticle after particle formulation. In other cases, the mitochondrial targeting agents are lipophilic cations that are covalently bound to the particle surface. In some cases, a cationic polymer is incorporated into the particle to target the particle to the mitochondrion.

### V. Methods of Determining Efficacy of Treatment

Alk1-cKO mice can also be used to determine the efficacy of potential therapeutics. For example, a potential therapeutic for treatment of AVM is administered to a first Alk1-cKO mouse. After a period of time, for example one or more hours, days, weeks, months, or years, the first Alk1-cKO mouse is compared to a second, control Alk1-cKO mouse which was not administered the therapeutic. The first and second Alk1-cKO mice are analyzed for development of AVM, expression of angiogenic or pro-inflammatory factors, or other symptoms of AVM such as AVM formation or hemorrhaging. Prevention or reduction of the development of AVM, expression of angiogenic or pro-inflammatory factors, or hemorrhaging in the first Alk1-cKO mouse, compared to the second, control Alk1-cKO mouse indicates an efficacious therapeutic. Methods for analyzing biological samples from Alk1-cKO are described above and in the Examples below. Methods for grading the severity of AVM are also described in the Examples below, see for example Tables 1 and 2, or are known in the art, see J. Neurosurg. Vol. (1986).

### VI. Pharmaceutical Compositions

As described in the Examples below, potential targets for therapeutic treatment of AVM, and/or prevention or reduction of symptoms of AVM such as AVM-associated hemorrhaging, include, but are not limited to, inhibitors of angiogenic and pro-inflammatory factors. In the preferred embodiments, the pharmaceutical compositions inhibit or decrease expression of VEGF, Cox-2, Agpt2, IL1β, TNF-α, or a matrix metalloproteinase. In one preferred embodiment, the pharmaceutical composition includes a Cox-2 inhibitor such as celecoxib. In another preferred embodiment the pharmaceutical composition includes a matrix metalloproteinase inhibitor such as doxycycline (DOX) or minocycline. Angiogenic and/or inflammatory factor inhibitors can be administered alone or in combination, and optionally include a pharmaceutically acceptable carrier for administration. Pharmaceutical compositions can be administered to subject in need thereof, such as a patient with AVM, using methods that are known in the art. Pharmaceutical compositions are typically administered in an effective amount to prevent or reduce one or more symptoms of AVM, for example, AVM formation, hemorrhaging, or elevated expression of one or more angiogenic or pro-inflammatory factors.

A. Pharmaceutically Acceptable Carriers

The compositions disclosed herein can be used prophylactically and therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

i. Liposomes

Pharmaceutical composition having an effective amount of one or more peptides can be carried in a liposome. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 .mu.m. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and are made of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 .mu.m. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 .mu.m, are described in Callo, et al., Cryobiology 22(3):251-267 (1985).

Mezei, et al., U.S. Pat. Nos. 4,485,054 and 4,761,288 also describe methods of preparing lipid vesicles, More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the disclosed compositions into liposomes. Generally, the fatty acid is a polar lipid. Thus, the fatty acid can be a phospholipid. The provided compositions can include either natural or synthetic phospholipid. The phospholipids can be selected from phospholipids containing saturated or unsaturated mono or disubstituted fatty acids and combinations thereof. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can include palmitoyl 16:0.

ii. Nanoparticles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

The nanoparticles can carry the AVM therapeutic. In some instances, the nanoparticle can be coated with a targeting agent that targets ischemic tissue.

### EXAMPLES

### Example 1

### Generation and Postnatal Lethality of Alk1-cKO Mice

### Materials and Methods

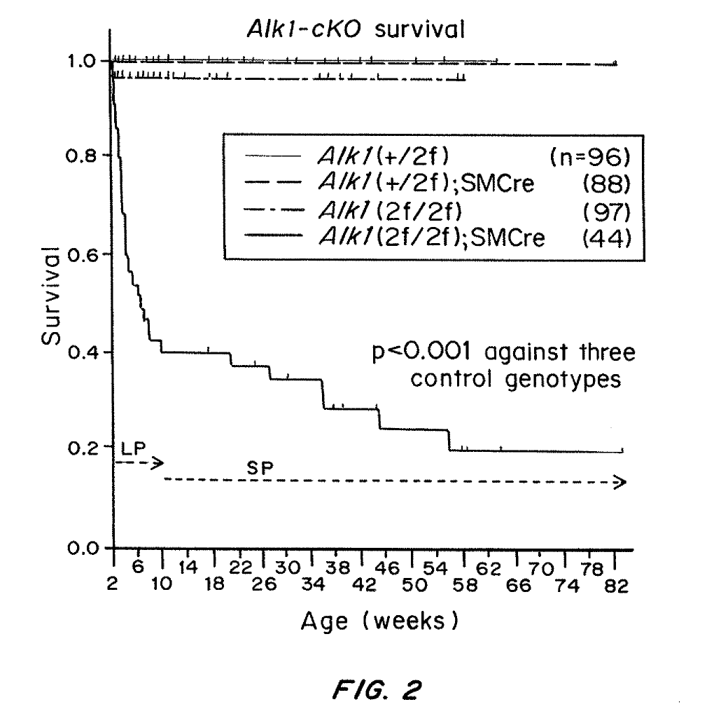

Alk1-cKO mice were generated by intercrossing Alk1 (+/−) heterozygous mice, Alk1(flox/flox) conditional deletion mice, and Tg(SM22-Cre) delete mice (Jackson Laboratory stock #004746). Alk1(flox/flox) mice have been utilized previously with other Cre-deleter mouse lines and showed 100% lethal phenotype due to severe internal hemorrhages. Both two cKO genotypes, Alk1(flox/flox);Tg(SM22-Cre) and Alk1(flox/−);Tg(SM22-Cre), lack functional Alk1 gene in the smooth muscle cells (SMCs) due to early embryonic Cre recombination by Tg(SM22-Cre) transgene. These two genotypes showed similar survival curves (p=0,31) and indistinguishable AVM phenotypes after 2 w of age. In addition, there was no difference in survival curve and phenotype between male and female cKOs. More than half of these mice were paralyzed or died due to internal hemorrhages during LP (data not shown) and the cerebellum was almost completely absorbed due to hemorrhage. The mice that survived into SP showed much improved survival rate (FIG. 2).

### Results

A majority of Alk1-cKO mice were paralyzed or died by 10 w of age due to hemorrhages in intracranial, spinal, and/or abdominal cavities (data not shown). Intriguingly, the mice surviving this early lethal period (LP, <10 w) did so in spite of carrying multiple AVMs in their brains and spinal cords (FIG. 2). Furthermore, preliminary longitudinal magnetic resonance (MR) angiography observation revealed that gross morphologies, relative sizes, and numbers of cerebral AVMs were largely unchanged in 3 Alk1-cKO mice after 3 w of age, indicating that most AVMs ceased growth by 3 w of age. Overall, these data show that mouse neurological tissues in LP may have a specific molecular and/or physiological environment that promotes hemorrhage from the AVM and it diminishes once they reached a stable period (SP, >10 w). The predominance of neurological AVM with partial lethality allows for several weeks of both mechanistic studies and therapeutic intervention.

Pathological Analysis of Alk1-cKO Mice

Almost all mice in SP were found with multiple AVMs and tortuous vessels (TVs) in the brain and/or spinal cords (data not shown). AVMs/TVs were visualized by injecting blue latex rubber dye (Connecticut Valley Biological Supply Company) through the cardiac left ventricle and subsequent tissue clearing by dehydration and methyl salicylate immersion. Due to the size of latex particles, the latex does not go into microcapillaries and only arterial vessels and AVMs/TVs are filled. The artery tree structure in littermate control mice using this blue latex was comparable to the one visualized by MR imaging. Many of the AVMs/TVs in Alk1-cKO mice were surrounded by hemosiderin deposition, illustrating history of prior hemorrhage (data not shown). The AVM/TV images were utilized to score severity of AVM using a novel mouse AVM grading system, which resembled the structure of the Spetzler-Martin grading system for human AVM by having three independent factors to determine the severity. In mice, number of AVMs/TVs, irregularity in diameter, and size of the biggest AVM/TV were independently scored and added up to have a score from 0 to 6 (Table 1).

TABLE 1
Graded features and scores in mouse AVM grading system

| | |
|---|---|
| Graded Feature | Point Assigned |

Size: size of the biggest AVM/TV
(size of mouse brain 10-15 mm in length)

| | |
|---|---|
| small: <0.5 mm | 0 |
| medium: 0.5-1.5 mm | 1 |
| large: >1.5 mm | 2 |

DI: diameter irregularity in any of the AVM/TV

| | |
|---|---|
| negative | 0 |
| positive | 1 |

Number of AVM/TV

| | |
|---|---|
| no lesion | 0 |
| 1 or 2 | 1 |
| 3-5 | 2 |
| >5 | 3 |

Score range: 0-6

A score 0 indicates no AVM/TV. Since hemorrhage is a critical indicator of severity, AVMs/TVs were scored with and without adjacent hemosiderin deposition separately (AVM/TV/+H and AVM/TV/−H, respectively). AVM/TV/+H and AVM/TV/−H scores were determined for each organ, resulting in four separate scores from each mouse, a set from the brain and another from the spinal cord (Table 2).

TABLE 2
Four AVM scores from a mouse

| Scores from single mouse | Score range |
|---|---|
| Brain AVM/TV/+H | 0-6 |
| Brain AVM/TV/−H | 0-6 |
| Spinal cord AVM/TV/+H | 0-6 |
| Spinal cord AVM/TV/−H | 0-6 |

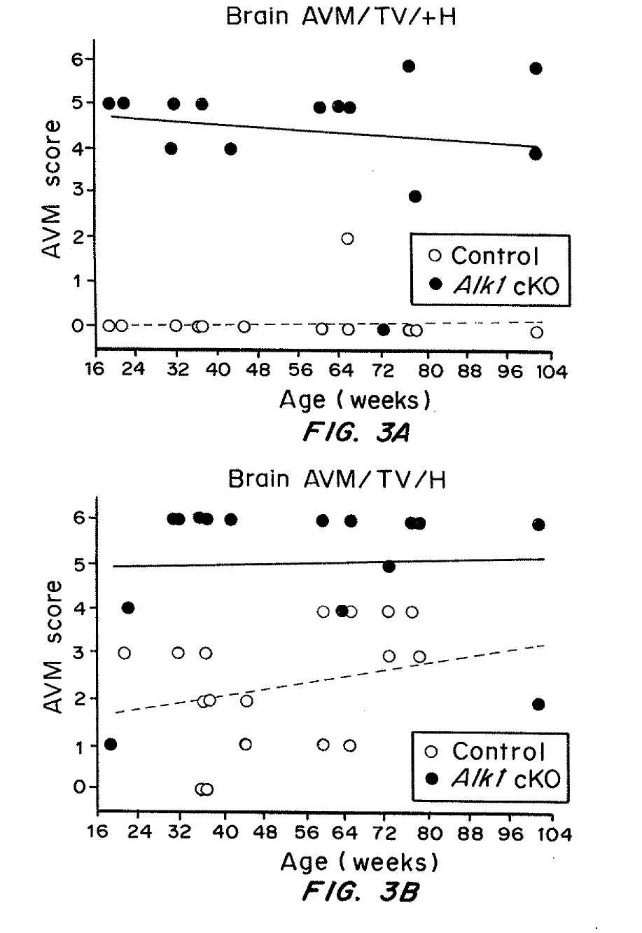

In the brain, plots of AVM/TV score over mouse age showed that 15 Alk1-cKO mice had consistently high scores for both AVM/TV/+H and AVM/TV/−H throughout SP (FIG. 3). In the spinal cord, most mice scored 0 for AVM/TV/+H (data not shown). This may be due to prior removal of paralyzed animals during LP, since hemorrhage in the spinal column generally causes hindlimb paralysis in Alk1-cKO mice. In the spinal cord, AVM/TV/−H scores showed 3 or 4 in a majority of Alk1-cKO mice (data not shown). In contrast, most AVM/TV scores for control littermates were 0 in brain and spinal cord, except in brain AVM/TV/−H, where it showed a trend of gradual increase due to an increase in number of small tortuous vessels. The consistently high AVM/TV scores in Alk1-cKO mice in the stable period (SP) age indicate that the mice developed a number of AVMs/TVs and experienced hemorrhage during the lethal period (LP).

Molecular Analysis of Alk1-cKO

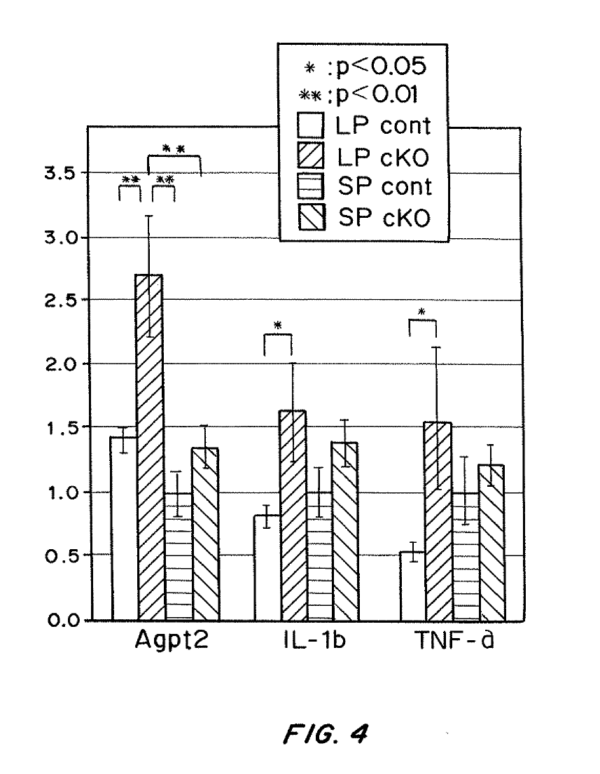

Although Alk1-cKO mice carried a number of AVMs in SP, these AVMs did not cause lethal hemorrhages, suggesting that the molecular and/or physiological environment in neurological tissues responsible for hemorrhage in LP has diminished in SP. In order to determine differences in general molecular environment in the brain, various angiogenic, inflammatory, extracellular molecule degradation, Alk1 signaling pathway, and vascular malformation related genes were screened using quantitative polymerase chain reaction (qPCR). The brain tissues (n=6 per group) used were not screened for AVMs and may or may not have contained AVMs. Among the 31 genes tested, the following 3 genes showed statistically significant differences between Alk1-cKO and control groups: angiopoietin 2 (Agpt2;ANGPT2), interleukin-1β (IL1β;IL1B), and tumor necrosis factor α (TNF-α;TNF) (FIG. 4). Immunohistochemical (IHC) staining of LP brain tissues confirmed the upregulation of Agpt2 and IL1β in AVM walls (data not shown). In addition, IHC staining identified upregulation of a few more angiogenic and inflammatory proteins, including vascular endothelial growth factor (VEGF;VEGFA) and cyclooxygenase-2 (Cox2;PTGS2) (data not shown). Intriguingly, similar to SMCs marked by anti-smooth muscle α actin (SMA;ACTA2) antibody, these proteins were expressed in a non-continuous, variegated pattern, showing a pathological nature of these vessels. Upregulation of inflammatory cytokines and Cox2 strongly indicates active inflammation in AVMs. Based on these findings, it is believed that an initial local tissue angiogenic/inflammatory insult causes angiogenic/inflammatory factor expression in vascular walls, triggering AVM development, weakening of vessels walls, and in some cases bleeding.

### Example 2

### Local Angiogenic/Inflammatory Stimulus in Skin Induces AVM Formation in ALK1 Conditional Deletion Mice

In human HHT patients, AVMs occur in only limited vascular beds and even the family members who share the same mutation develop AVMs in different organs. Similarly, even though most of the SMCs lacked functional Alk1 gene in Alk1-cKO mice, only limited vascular beds developed AVMs/TVs. For instance, most Alk1-cKO mice developed AVMs/TVs in the central nervous system, but skin was rarely found with malformed vessels and never caused hemorrhages.

Local angiogenic and/or inflammatory stimulus was proposed to be the trigger of AVM development and used to induce malformed vessels in mice. Adenoviral expression of VEGF in Eng mutant mice caused tortuous capillaries in the brain, which did not grow bigger or bleed. Inflammation due to skin wound caused acute skin AVM formation in postnatally induced whole-body Alk1 deletion, however, in this case the mice were severely ill due to hypoxia, intestinal bleeding, and anemia.

Acute Inflammation Triggered by Skin Wound Induces AVM Development

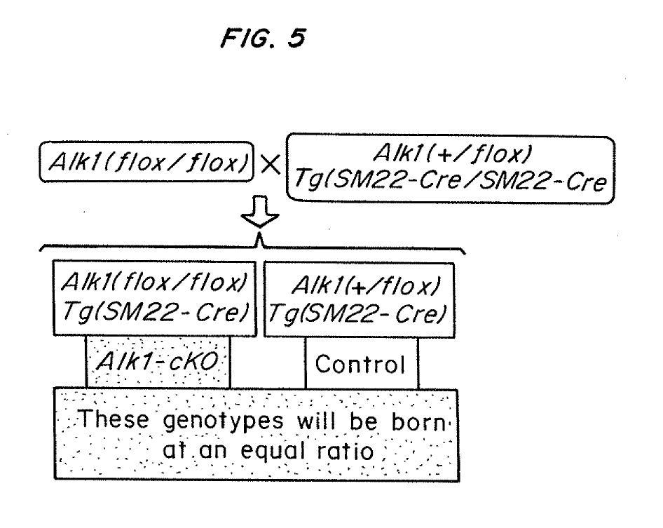

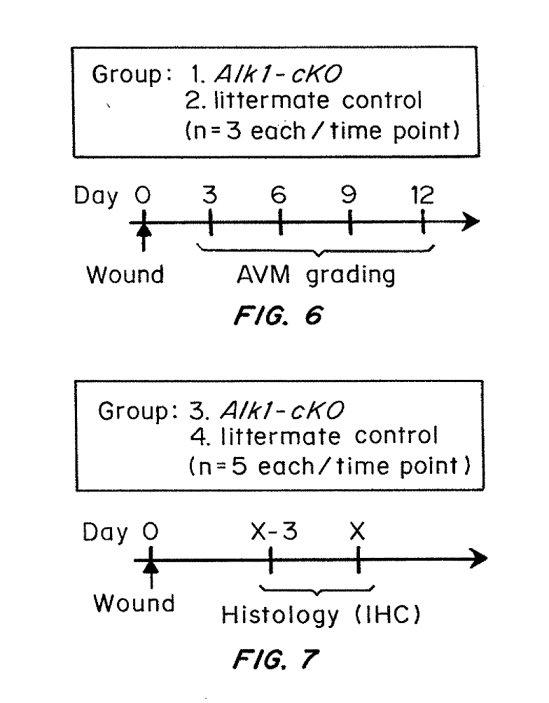

Strategies: Experiment mice are generated by mating Alk1(flox/flox) and Alk1(+/flox);Tg(SM22-Cre/SM22-Cre) mice in the existing breeding colonies (FIG. 5). The latter mice are homozygous for SM22-Cre transgene and therefore the mating generates Alk1-cKO and its littermate control at 1:1 ratio, ensuring adequate supply of experiment animals. Both male and female mice are utilized since there was no difference in their survival and phenotype. Full-thickness skin wound are created on the back of Alk1-cKO and littermate control mice at 11 w of age as previously performed (FIG. 6). The mice at this age are in SP and not prone to hemorrhage from AVMs. During the 2-week wound healing period in mice, the wound induces both angiogenic and inflammatory factors in the surrounding skin tissue.

Mice at 3, 6, 9, and 12 days after wounding (n=3 per group/time point) are injected with the blue latex solution into the cardiac left ventricle prior to skin removal for AVM visualization (data not shown). The skin samples are observed under microscopy and digital photographs are recorded. Severity of AVMs/TVs re determined for each mouse using the mouse AVM grading system (Table 1). Once the time point that AVMs/TVs start to form is determined, another set of wounded mice (n=5 per genotype/time point) are generated for histological analyses (FIG. 7). Tissue samples are collected without latex injection at the time points just prior to and at the point of AVM formation and the sections are IHC stained with Agpt2, VEGF, IL1β, and Cox2 antibodies to examine their expression in vasculature. Platelet endothelial cell adhesion molecule (PECAM) and SMA antibodies are utilized to distinguish endothelial cell (ECs) and SMCs in the tissue.

Skin tissues near and distant (abdominal-side skin) from the wound lesion are compared for expression of angiogenic/inflammatory genes. More genes may be IHC stained once molecular analyses in following examples identify additional candidate angiogenic/inflammatory genes involved in AVM pathogenesis.

Statistics: AVM scores are compared using Mann-Whitney U Test between Groups 1/2. The number of IHC stained vessels are analyzed by analysis of variance (ANOVA) followed by an appropriate post hoc test between near and distant skin samples in both groups.

Up-Regulation of Vascular Endothelial Growth Factor (VEGF) Induces Skin AVM Using VEGF Transgenic Mice

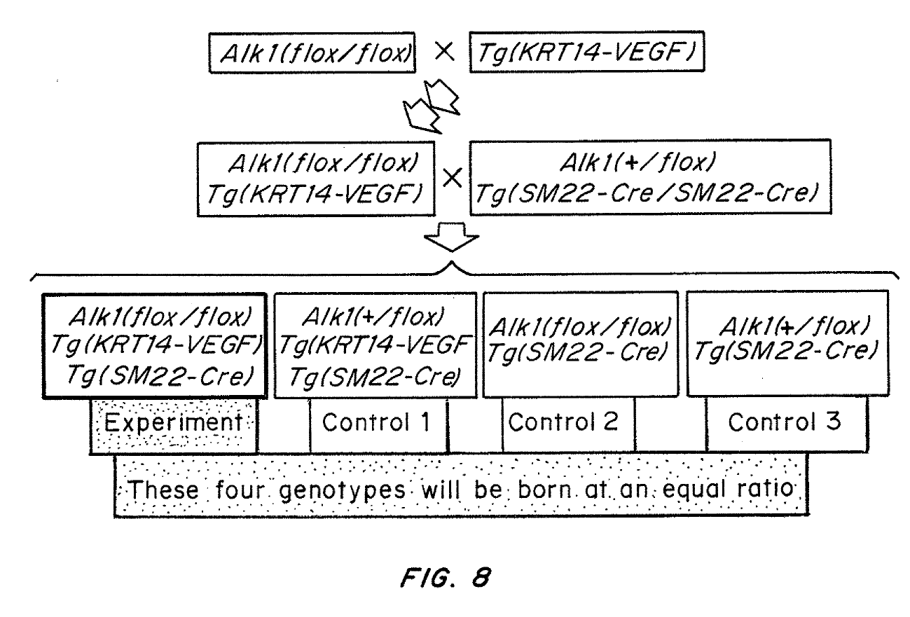

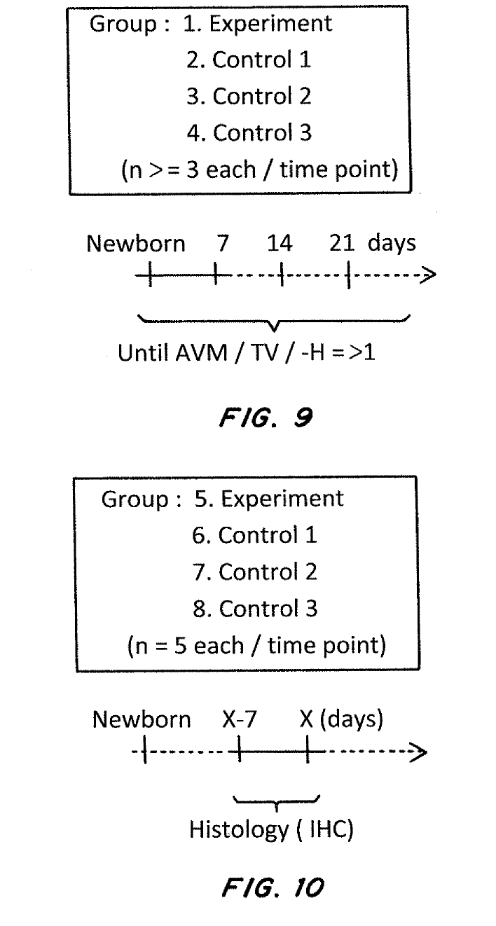

Strategies: Experiment mice are generated by first crossing Alk1(flox/flox) and Tg(KRT14-VEGF) mouse lines (Jackson Laboratory stock #: 005705) to generate Alk1(flox/flox);Tg(KRT14-VEGF) mice. The Alk1(flox/flox);Tg(KRT14-VEGF) mice are then mated to existing Alk1(+/flox);Tg(SM22-Cre/SM22-Cre) mice to generate the experiment and littermate control mice (FIG. 8). One out of four pups from the mating are the experiment genotype. Both male and female mice are utilized. Tg(KRT14-VEGF) mice are viable and fertile. In these mice, the transgene induces chronic VEGF (active 164 amino acid isoform) expression in skin keratinocytes due to keratin 14 (KRT14) promoter in the construct. The mice show a consistent increase in skin microvascular density (MVD) for at least 6 weeks postnatally with the highest density at a neonate stage. The mice also show an increase in leukocyte rolling and adhesion in skin microvessels and hence demonstrate key features of chronic skin inflammation. Experiment and littermate control newborn pups are searched for AVMs/TVs in the skin using the blue latex injection (FIG. 9).

Since the Tg(KRT14-VEGF) mice show the highest MVD in neonates, searching AVM/TV formation starts at from this age and continues at 1 week intervals (n=>3 each per time point) until all experiment mice show 1 or higher AVM/TV/−H score (Table 1) or until 10 w of age. Once the time point is determined, another set of mice (n=5 per genotype/time points) are sacrificed at the time points just prior to and at the point of the AVM/TV formation without latex injection, and the skin tissues is used for histological analyses as described in above (FIG. 10). In addition, MVD is determined by counting the number of PECAM stained capillaries per view filed.

Statistics: AVM scores and MVD are analyzed by ANOVA followed by a post hoc test.

### Example 3

### Cyclooxygenase-2 Inhibitor Celecoxib Reduces Hemorrhage from AVM

Rationale: Although many patients who carry AVMs are in need of a drug that reduces the chance of hemorrhage, there are currently none available. In human AVMs, involvement of inflammatory cells in the lesion is reported. In AVM walls in Alk1-cKO mice, variegated expression of inflammation related molecules including TNF-α, IL1β, and Cox2 were observed (FIG. 4 and data not shown), indicating active inflammatory reaction in the lesion. Cox2 is an inducible enzyme, which plays a key role in inflammatory process, produces prostaglandins, and activates a variety of downstream inflammatory pathways. Since Cox2 inhibition by select inhibitor celecoxib significantly reduces tissue inflammatory response including tissue damage and remodeling, celecoxib is utilized in this example. Furthermore, select Cox2 inhibitors have an advantage over non-select Cox inhibitors in AVM treatment, since the common non-select Cox inhibitors, like aspirin and ibuprofen, have an adverse effect of decreasing plate aggregation through Cox1 inhibition, which may potentially escalate hemorrhagic events. Celecoxib was administered to Alk1-cKO mice during LP and changes in number/severity of hemorrhagic incidence as well as histological and molecular changes in AVM tissues were observed.

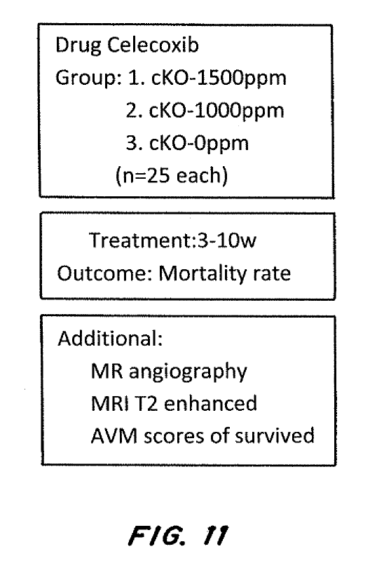

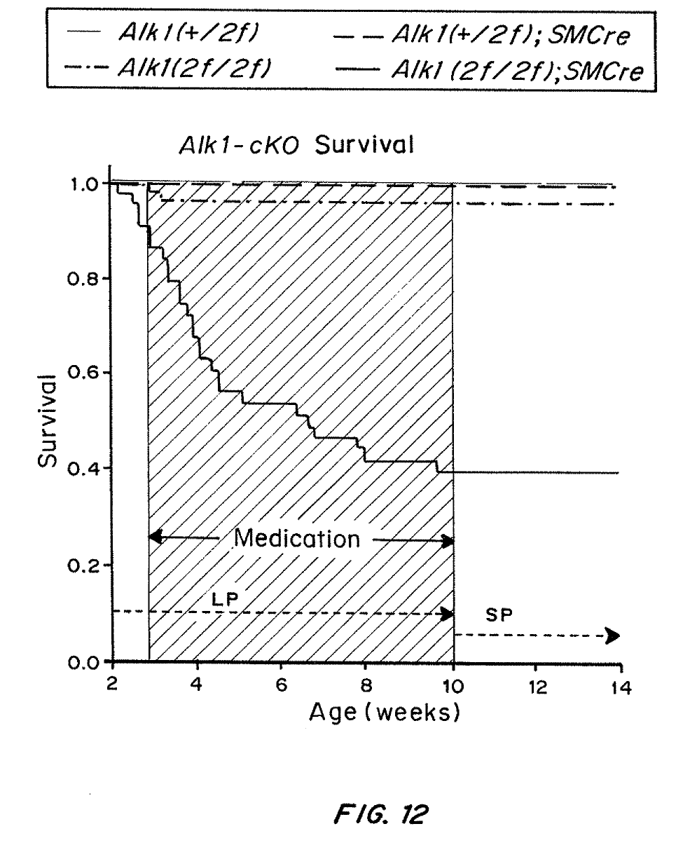

Strategies: Alk1-cKO and littermate control mice are generated as described above (FIG. 5). Alk1-cKO mice are genotyped at 2 w of age, weaned at 3 w, and separated into 3 diet groups (n=25 each) (FIG. 11). The medicated mouse diets are generated by supplementing standard rodent chow with 1000 and 1500 mg/kg (1000 and 1500 ppm) of select Cox2 inhibitor celecoxib (Sigma) by Harlan Laboratories. These celecoxib doses are commonly utilized, well tolerated, and effective in Cox2 inhibition in mice. The mice were fed with the chows ad libitum until the end of LP (10 w of age, FIG. 12), when any remaining mice are subjected to AVM grading. The primary outcome of this drug test is a change in mortality rate at the end of treatment. During the course of treatment, mouse health conditions are observed daily and any paralyzed mice are removed for AVM grading, Necropsy is performed to identify any internal bleeding of dead mice.

In addition, changes in AVM morphology in the subset of mice (n=10 per group) is tracked during the drug treatment using MR imaging for both the brain and spinal cord. At 3 w, 6 w, and 10 w of age, MR imaging is performed using a Bruker 7T 20 cm BioSpec MR spectrometer with a standard transmit/receive volume coil (35 mm i.d.) (Bruker Instruments). Mice are anesthetized, positioned on a cradle to maintain a constant body temperature (37.8° C.), and secured by medical tape. ECG and respiratory signals are monitored. MR angiography images are collected to locate AVMs using a flow-compensated, 2D time-of-flight sequence (data not shown).

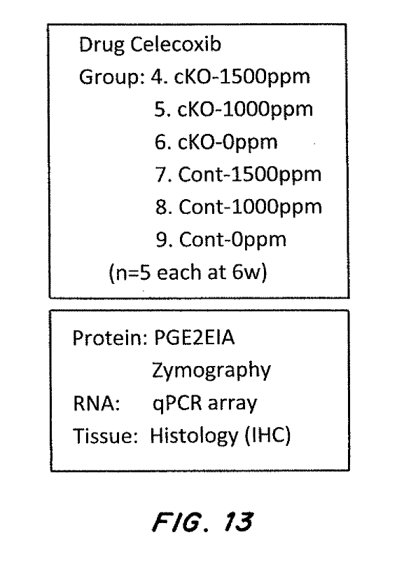

T2*-weighted images are acquired to determine the location, number, and severity of hemorrhage using a gradient echo sequence coregistered to the MRA image volume, which is standard in neuroradiological exams in human (data not shown). The severity of hemorrhage are assessed using the volume of T2* signal decrease as a proxy. At the end of drug treatment, any surviving mice are injected with blue latex and AVM grading are performed (Table 1). The grading includes separate scoring for AVMs with and without hemorrhages (AVM/TV/+H and AVM/TV/−H, respectably). The brain and spinal cord are scored separately (Table 2). In addition, to confirm the efficacy of drug treatment, a separate set of Alk1-cKO groups (n=5 each) are scanned with MR imaging and killed at 6 w of age for molecular and histological analyses (FIG. 13). Each AVM and corresponding control brain tissue are collected, divided into 3 pieces, and used for protein, RNA, and histology sample preparations. Prepared protein are used for quantification of the major Cox2 product, prostaglandin E2 (PGE2), with PGE2 enzyme immunoassay (EIA) kit (Cayman Chemical). Also, the protein is used for gelatin zymography (Invitrogen) to assess the MMP activity. The zymography results are photographed and band intensities are quantified using the ImageJ program.

The RNA samples are used to confirm changes in Cox2 downstream gene expression by running qPCR arrays. Potential Cox2 downstream genes encompass a number of inflammatory factors including IL10, IL12, IL6, chemokine (C—C motif) ligand 2 (CCL2), CCL4, and C—X—C motif chemokine 10 (CXCL10), 27,28 as well as angiogenesis related genes including VEGF, hypoxiainducible factor 1 α subunit (HIF-1), integrin αv, integrin β3, MMP2, and MMP9. In order to assess the expression levels of these and other angiogenic/inflammatory genes, the Inflammatory Response & Autoimmunity PCR Array and Endothelial Cell Biology PCR Array (both from Qiagen) is used to perform relative quantification (ΔCt method) of more than 160 genes. The analysis is conducted using preassembled 96-well plate arrays, because it is fast, comprehensive, and economical. Individual Taqman assays can also be used. Furthermore, because of the extensive number of genes on arrays, additional treatment target genes that play a key role in AVM hemorrhage are identified in this way.

The histological sections are IHC stained with anti-VEGF, Agpt2, IL1β, Cox2, PECAM, and SMA antibodies (data not shown). Moreover, antibodies against the additional key genes identified by qPCR arrays are used to identify their localization in the brain tissues by IHC. Antibodies against most genes in the arrays are commercially available.

Statistics: Mouse mortality rate is compared using z-test between Group 1 and 3 as well as Group 2 and 3. The AVM scores, PGE2 EIA, zymography intensities, and differences in ΔΔCt values in the qPCR data is analyzed by ANOVA followed by a post hoc test. IHC stained slides is photographed and a ratio between the lengths of AVM circumference and positively stained parts are determined using the ImageJ program. For each protein, the IHC positive ratios in Groups 4, 5, and 6 are analyzed by ANOVA followed by a post hoc test.

### Example 4

### Matrix Metalloproteinase Inhibitor Doxycycline Reduces Hemorrhage from AVM

Rationale: Elevated activity of MMPs in human AVM lesion and AVM patient's blood has been reported. Activated MMPs can degrade vascular wall and precede to expansion of vascular aneurysm in animal model. MMPs may be involved in AVM pathogenesis and potentially in its spontaneous rupture. Tetracycline derivatives, including doxycycline (DOX) and minocycline, have nonspecific MMP inhibitory effects that are distinct from their antimicrobial action. This anti-MMP effect has been shown in vivo in mouse brain and ex vivo in human AVM tissues. Furthermore, these drugs appear to have anti-inflammatory properties in addition to anti-MMP effects. These and other growing evidences lead to an ongoing human clinical trial, where tetracyclines are administered to AVM patients to stabilize AVM walls and hence prevent spontaneous hemorrhage. Like in human AVM, elevated MMP activity was observed in a few of AVM lesions in Alk1-cKO mice, which may have caused the prior hemorrhage from the AVMs or would have caused the next one (data not shown). DOX is administered to Alk1-cKO mice during LP to inhibit MMPs and observe changes in number/severity of hemorrhagic incidence as well as histological and molecular changes in AVM tissues.

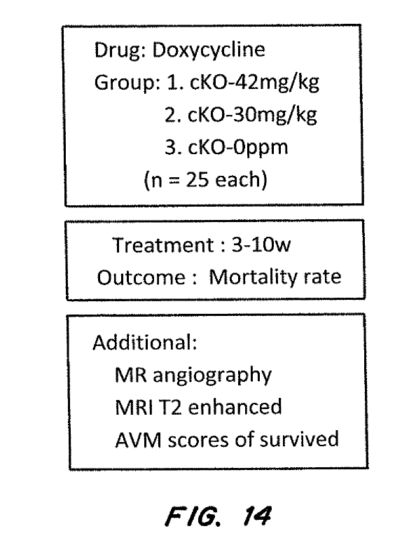

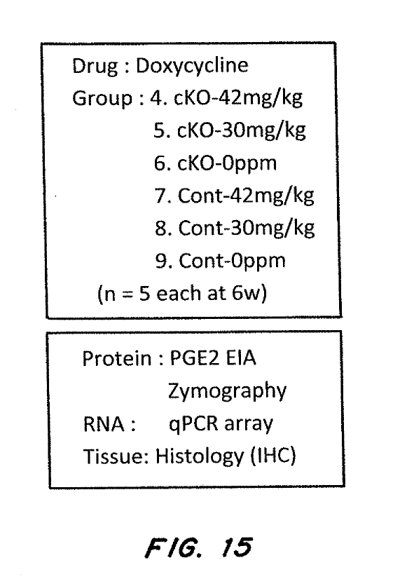

Strategies: A design of experiments in this example is essentially identical to those described above for Example 3. DOX (Sigma) is administered daily in the drinking water at doses of 30 and 42 mg/kg per day (based on the average daily water consumption, 170 and 240 mg/L in the drinking water, respectively). The 30 mg/kg per day dose has shown to inhibit MMP activity in mouse brain and aorta. The 42 mg/kg per day dose has successfully prevented aortic aneurysm in Malfan syndrome model mice. Various measurements of drug treatment outcomes are carried out as described in Example 3 (FIG. 14 and FIG. 15)

### Example 5

### Age-Dependent Lethality in Novel Transgenic Mouse Models of Central Nervous System Arteriovenous Malformations

### Rationale

Hemorrhage from AVMs in the CNS may cause severe neurological deficit or death. Limited research in the development of therapeutic medications for AVMs has been due, in a large part, to an absence of an appropriate animal model. In humans, a pathological ALK1 mutation causes hereditary hemorrhagic telangiectasia (HHT), a disease characterized by AVM formation in the CNS and other organs. Although AVM mouse models have been generated by conventional and conditional Alk1 deletions, all models caused severe vascular malformations in diverse organs and inevitable lethal hemorrhages (Oh S P, et al. Activin receptor-like kinase 1 modulates transforming growth factor-beta 1 signaling in the regulation of angiogenesis. Proc Natl Acad Sci USA. 2000; 97:2626-2631; Seki T, Yun J, Oh S P. Arterial endothelium-specific activin receptor-like kinase 1 expression suggests its role in arterialization and vascular remodeling. Circulation research. 2003; 93:682-689; Park S O, et al. Alk5- and tgfbr2-independent role of alk1 in the pathogenesis of hereditary hemorrhagic telangiectasia type 2. Blood. 2008; 111:633-642; Park S O, et al. Real-time imaging of de novo arteriovenous malformation in a mouse model of hereditary hemorrhagic telangiectasia. J Clin Invest. 2009; 119:3487-3496). Recently, a mouse brain AVM model was developed by a viral vector-mediated method using the Alk1 conditional deletion mice, however, this model does not cause hemorrhage or neurological deficit (Walker E J, et al. Arteriovenous malformation in the adult mouse brain resembling the human disease. Annals of neurology. 2011; 69:954-962).

The present data show CNS AVM mouse models exhibiting hemorrhage, paralysis, and partial lethality. All of these mice developed AVMs in their brains, and a majority of them died or were paralyzed due to internal hemorrhages before reaching 10 weeks of age. However, a subset of mice survived much longer despite carrying multiple AVMs. In addition, variegated expression of angiopoietin 2 (Agpt2) and a few inflammation related genes in the rupture-prone AVM walls were identified.

### Materials and Methods

Animals and

All mouse procedures carried out were reviewed and approved by the Georgia Health Sciences University (GHSU) Institutional Animal Care and Use Committee. Floxed-Alk1 conditional deletion mice Alk1 (flox/flox) (Park S O, et al. Alk5- and tgfbr2-independent role of alk1 in the pathogenesis of hereditary hemorrhagic telangiectasia type 2. Blood. 2008; 111:633-642; Park S O, et al. Real-time imaging of de novo arteriovenous malformation in a mouse model of hereditary hemorrhagic telangiectasia. J Clin Invest. 2009; 119:3487-3496) Alk1 deletion mice Alk1 (+/−),2 and transgenic Tg(SM22-Cre) deleter mice (Jackson Laboratory) (Holtwick R, et al. Smooth muscleselective deletion of guanylyl cyclase-a prevents the acute but not chronic effects of anp on blood pressure. Proc Natl Acad Sci USA. 2002; 99:7142-7147) were intercrossed to generate SM22Cre-del models. For identification of Cre-recombined cells, R26R Cre-reporter mice (Jackson Laboratory) (Soriano P. Generalized lacz expression with the rosa26 cre reporter strain. Nat Genet. 1999; 21:70-71) were crossed with the above mouse lines. Littermate control mice were used for all experiments.

The Alk1 (+/flox) and Alk1(+/−) mice were developed originally in 129Sv genetic background, however backcrossed to C57BL/6 genetic background for several generations before intercrosses. The Tg(SM22-Cre) were generated in B6SJLF2 background and maintained by sister x brother cross in the Jackson Laboratory. R26R mice were backcrossed to C57BL6/J background for more than 10 generations in the Jackson Laboratory prior to the purchase. Therefore, the SM22Cre-del mice used in the experiments had mixed genetic background of the above genetically modified mouse lines. All experiments were carried out with littermate control mice to regulate genetic background effect.

The offspring was genotyped at 2 weeks of age by polymerase chain reaction (PCR) using tail DNAs. Genotyping with a primer set specific for the Alk1 null allele (3F and 6R primers reported by Park et al) (Park S O, et al. Alk5- and tgfbr2-independent role of alk1 in the pathogenesis of hereditary hemorrhagic telangiectasia type 2. Blood. 2008; 111:633-642) showed a presence of Cre-recombined Alk1-null allele in SM22Cre-del tails, confirming a successful recombination of Alk1-flox allele by the SM22-Cre transgene (data not shown).

Paralyzed animals were euthanized promptly since they often showed difficulty in feeding. Animals found dead were undergone necropsy, and most necropsy specimens appeared to show signs of significant hemorrhage in multiple internal cavities in partially decomposed bodies (data not shown). However, it is unclear whether it is postmortem artifact or an actual cause of death since comparable hemorrhagic lesions were identified in a few of the similarly decomposing littermate control mouse bodies.

Blue Latex Injection

Blue latex solution was systemically injected through the cardiac left ventricle, and tissues were imaged as previously described (Park S O, et al. Alk5- and tgfbr2-independent role of alk1 in the pathogenesis of hereditary hemorrhagic telangiectasia type 2. Blood. 2008; 111:633-642; Park S O, et al. Real-time imaging of de novo arteriovenous malformation in a mouse model of hereditary hemorrhagic telangiectasia. J Clin Invest. 2009; 119:3487-3496). Briefly, 2 mL of the blue latex solution was diluted with 1 mL of 0.9% NaCl or phosphate buffered saline (PBS) solution. A mouse was anesthetized with intra peritoneal (i.p.) injection of tribromoethanol (Avertin), and its chest cavity was exposed by thoracotomy. Following heparin perfusion through cardiac left ventricle, the diluted blue latex was perfused through the left ventricle to complete the vascular casting. The inferior vena cava (IVC) was cut during the heparin perfusion for bleeding.

Subsequent to an examination of arteriovenous malformation (AVM) incidence in abdominal cavity under microscopy, brain and spinal column tissues were collected, fixed with formalin, and dehydrated with methanol series (Park S O, et al. Alk5- and tgfbr2-independent role of alk1 in the pathogenesis of hereditary hemorrhagic telangiectasia type 2. Blood. 2008; 111:633-642). The tissues were then cleared with 100% methyl salicylate (Sigma-Aldrich) and photographed. Since the latex cannot enter microcapillaries due to its particle size, AVM search could not be performed in some of the major organs, including the lungs and liver.

Relative Quantitative PCR (qPCR)

Total RNAs from the most AVM-prone segment of the cerebral hemisphere, roughly right striatum and basal ganglia regions, were isolated from four groups of animals (n=6 each): lethal period (LP) Flox-SM22Cre-del mice (4-8 w of age), LP littermate control mice, stable period (SP) SM22Cre-del mice (29-36 w of age), and SP littermate control mice. TaqMan Gene Expression Assays (Applied Biosystems) was utilized for the relative quantification of transcripts, and results are shown as fold difference compared to the SP control.

Based on the previous vascular malformation-related studies (Holtwick R, et al. Smooth muscleselective deletion of guanylyl cyclase-a prevents the acute but not chronic effects of anp on blood pressure. Proc Natl Acad Sci USA. 2002; 99:7142-7147; Soriano P. Generalized lacz expression with the rosa26 ere reporter strain. Nat Genet. 1999; 21:70-71; Chen Y, et al. Evidence of inflammatory cell involvement in brain arteriovenous malformations. Neurosurgery. 2008; 62:1340-1349; discussion 1349-1350; Kim H,et al. Brain arteriovenous malformation pathogenesis: A response-to-injury paradigm. In: Zhang J, Colohan A, eds. Intracerebral hemorrhage research. Springer; 2011: 83-92; Hashimoto T, et al. Abnormal balance in the angiopoietin-tie2 system in human brain arteriovenous malformations. Circulation research. 2001; 89:111-113; Chen Y, et al. Interleukin-6 involvement in brain arteriovenous malformations. Annals of neurology. 2006; 59:72-80; Sure U, et al. Endothelial proliferation, neoangiogenesis, and potential de novo generation of cerebrovascular malformations. Journal of neurosurgery. 2001; 94:972-977; Carlson T R, et al. Endothelial expression of constitutively active notch4 elicits reversible arteriovenous malformations in adult mice. Proceedings of the National Academy of Sciences of the United States of America. 2005; 102:9884-9889; ZhuGe Q, et al. Notch-1 signalling is activated in brain arteriovenous malformations in humans. Brain: a journal of neurology. 2009; 132:3231-3241; Tian X L, Kadaba R, You S A, Liu M, Timur A A, Yang L, et al. Identification of an angiogenic factor that when mutated causes susceptibility to klippel-trenaunay syndrome. Nature. 2004; 427:640-645; Kamada F, et al. A genomewide association study identifies rnf213 as the first moyamoya disease gene. Journal of human genetics. 2011; 56:34-40; Chan A C, et al. Recent insights into cerebral cavernous malformations: Animal models of ccm and the human phenotype. The FEBS journal. 2010; 277:1076-1083; Shovlin C L. Hereditary haemorrhagic telangiectasia: Pathophysiology, diagnosis and treatment. Blood reviews. 2010; 24:203-219), 31 genes associated with angiogenesis, extracellular matrix degradation, inflammation, vascular malformation, and Alk1 signaling pathways were selected for quantification. Relative quantification of transcripts with comparative ΔΔCt method was performed by following the manufacturer's recommended protocols using the Applied 3 Biosystems 7500 Fast Real-Time PCR Systems and the following pre-designed TaqMan Gene Expression Assays (Applied Biosystems): Agpt1, gene symbol: Angpt1 , gene name: angiopoietin 1, Assay ID: Mm01129232_m1; Agpt2, Angpt2, angiopoietin 2, Mm00545822_m1; Tie-1, Tie1, tyrosine kinase with immunoglobulin-like and EGF-like domains 1, Mm00441786_m1; PDGF-B , Pdgfb, platelet derived growth factor B polypeptide, Mm00440677_m1; bFGF, Fgf2, fibroblast growth factor 2, Mm00433287_m1; VEGF-A, Vegfa, vascular endothelial growth factor A, Mm00437306_m1; VEGF-B, Vegfb, vascular endothelial growth factor B, Mm00442102_m1; VG5Q, Aggf1, angiogenic factor with G patch and FHA domains 1, Mm00546486_m1; RNF213, Rnf213, ring finger protein 213, Mm00660047_m1; HIF-1α, Hif1α, hypoxia inducible factor 1, alpha subunit, Mm00468869_m1; MMP-2, Mmp2, matrix metallopeptidase 2, Mm00439505_m1; MMP-9, Mmp9, matrix metallopeptidase 9, Mm00600163_m1; TIMP-1, Timp1 , tissue inhibitor of metalloproteinase 1, Mm00441818_m1; TIMP-2, Timp2, tissue inhibitor of metalloproteinase 2, Mm00441825_m1; TIMP-3, Timp3, tissue inhibitor of metalloproteinase 3, Mm00441826_m1; TIMP-4, Timp4, tissue inhibitor of metalloproteinase 4, Mm00446568_m1; IL-6, Il6, interleukin 6, Mm99999064_m1; IL-1β, Il1b, interleukin 1 beta, Mm00434228_m1; TNF-α, Tnf, tumor necrosis factor, Mm99999068_m1; COX-2, Ptgs2, prostaglandinendoperoxide synthase 2, Mm00478374_m1; Krit-1, Krit1, KRIT1 ankyrin repeat containing, Mm00459502_m1; CCM2, Ccm2, cerebral cavernous malformation 2 homolog (human), Mm00524581_m1; Pdcd10, Pdcd10, programmed cell death 10, Mm00727342_s1, Alk1, Acvrl1, activin A receptor, type II-like 1, Mm00437432_m1; Eng, Eng, endoglin, Mm00468256_m1; Smad4, Smad4, MAD homolog 4 (Drosophila), Mm03023996_m1; Tmem100, Tmem100, transmembrane protein 100, Mm00471352_m1; BMP-9, Gdf2, growth differentiation factor 2, Mm00807340_m1; Dll-4, Dll4, deltalike 4 (Drosophila), Mm00444619_m1; Notch-1, Notch1, Notch gene homolog 1 (Drosophila), Mm00435245_m1; Notch-4, Notch4, Notch gene homolog 4 (Drosophila), Mm00440525_m1.

The RNA sample loading was normalized using the Ct value of 18S RNA assay (Catalog #: 4319413E, Applied Biosystems) and then fold difference compared to the SP control sample was calculated (ΔΔCt method).

Whole Mount Tissue X-Gal Staining

The cells undergone Cre recombination were visualized by crossing Tg(SM22-Cre) Cre-deleter line4 with R26R(+/lacZ) Cre-reporter line, 5 where Cre recombination causes reporter lacZ gene expression. Whole mount X-gal staining was performed as previously described (Park S O, et al. Alk5- and tgfbr2-independent role of alk1 in the pathogenesis of hereditary hemorrhagic telangiectasia type 2. Blood. 2008; 111:633-642). Briefly, fresh tissue samples were fixed in fixative solution consisting of 1% formaldehyde, 0.2% glutaraldehyde, 2 mM MgCl2, 5 mM ethylene glycol tetraacetic acid (EGTA), and 0.02% NP-40 in PBS for 10 min with gentle rocking. After rinsing with PBS twice, the tissue samples were then incubated in X-gal staining solution consisting of 5 mM potassium ferricyanide, 5 mM potassium ferrocyanide, 2 mM MgCl2, 0.01% sodium demoxycholate, 0.02% NP-40, and 0.75 mG/mL X-gal, in PBS overnight at 37° C. with gentle rocking. On the following day, the samples were rinsed with PBS twice and photographed.

Magnetic Resonance Angiography

Magnetic resonance (MR) imaging was performed using a Bruker 7T 20 cm BioSpec MR spectrometer with a standard transmit/receive volume coil (35 mm i.d.) (Bruker Instruments). Mice were anesthetized with isoflurane inhalation, positioned on a cradle to maintain a constant body temperature (37.8° C.), and secured using medical tape. Electrocardiogram and respiratory signals were monitored. MR angiography images were collected to locate AVMs using a flow-compensated, axial 2D time-of-flight sequence (TE/TR=4/17 msec; FOV=19×19 mm2; Acq Matrix=384×384; 80 slices; 0.2 mm slice thickness; 12 averages). Maximum intensity projections (MIP) of the MR angiography image volumes were constructed using the NIH ImageJ program (Abramoff M D, et al. Image processing with imagej. Biophotonics International. 2004; 11:36-42).

Histological Analyses

Localizations of brain AVMs were identified by magnetic resonance (MR) angiography, and tissues were collected without any cardiac perfusion. Colorimetric immunohistochemical (IHC) staining was performed to observe unpredictable AVM histology and localization of specific protein on a single section.

The following antibodies were used for immunohistochemical (IHC) staining: anti-smooth muscle α actin (SMA, dilution 1:800, catalog #A2547, Sigma-Aldrich), anti-angiopoietin 2 (Agpt2, 1:300,Ab65835, Abeam), anti-interleukin-1β (IL-1β, 1:800, NBP1-03300, Novus Biologicals), anti-platelet endothelial cell adhesion molecule (PECAM, 1:200, 550274, BD Pharmingen), anti-cyclooxygenase 2 (Cox2, 1:200, 160126, Cayman Chemical), anti-minichromosome maintenance complex component 7 (MCM7 also known as CDC47, 1:500, MS862P1, Lab Vision), and anti-proliferating cell nuclear antigen (PCNA, 1:6000, Ab29, Abcam).

Tissue samples for histological analyses were collected from Flox-SM22Cre-del and littermate control mice (n=4 mice each, 5-6 w of age) without any cardiac perfusion, followed by paraformaldehyde-fixation and paraffin-embedding, to preserve the fragile AVM tissue histology. Colorimetric IHC staining was performed since it enables us to observe unpredictable, irregular AVM histology and localization of specific protein on a single section. IHC staining was performed with VECTASTAIN Elite ABC Kit or Vector M.O.M. Peroxidase Kit along with ImmPACT DAB Peroxidase Substrate (Vector Laboratories), followed by hematoxylin counter staining. Antigen retrieval was performed for the following antibodies using Declere Pretreatment Solution and an electric pressure cooker (Cell Marque Corporation) by following the manufacturer's recommended protocol: Agpt2, IL-1β, Cox2, MCM7, and PCNA.

For double IHC staining, the sample slides were treated with hydrogen peroxide and Avidin/Biotin Blocking (Vector Laboratories) solutions after the completion of the first IHC to eliminate the crossreaction with the second IHC. Colorimetric reaction of the second IHC was performed using ImmPACT VIP Substrate. As a negative control, some of the sections were stained without the primary antibody for either the first or second (SMA) IHC. No counter staining was performed on these sections.

X-gal staining on frozen tissue samples was performed as described (Seki T, Yun J, Oh S P. Arterial endothelium-specific activin receptor-like kinase 1 expression suggests its role in arterialization and vascular remodeling. Circulation research. 2003; 93:682-689). Briefly, tissue samples were sequentially treated with paraformaldehyde (PFA) and sucrose solutions, embedded in OCT compound, sectioned, and X-gal stained overnight by following the published standard protocol (Nagy A, et al. Protocol 15: Staining for betagalactosidase (lacz) activity. In: Inglis J, Cuddihy J, eds. Manipulating the mouse embryo: A laboratory manual. Cold Spring Harbor, N.Y.: Cold Spring Harbor Laboratory Press; 2003: 687-691). X-gal stained sections were then either cover-slipped without counter-staining; counter-stained with nuclear fast red (Vector Laboratories) followed by cover-slipping; or IHC-stained without subsequent counter-staining, as described (Seki T, Yun J, Oh S P. Arterial endothelium-specific activin receptor-like kinase I expression suggests its role in arterialization and vascular remodeling. Circulation research. 2003; 93:682-689). In order to detect the slightest X-gal staining, the samples cover-slipped without counterstaining were carefully examined under microscopy for very weak X-gal signals, which generally appear as a tiny blue dot. Any signal detected were then matched and identified on the next serially-sectioned IHC-stained sample to determine if it colocalized with IHC signals.

Statistical Analyses

Statistical and survival analyses were performed using SigmaPlot 11.0 software (Systat Software Inc.). Proportions of genotypes in offspring were tested with chi-square test. The logrank significant test was performed for Kaplan-Meier survival curves. Delta-delta-Ct values from Taqman quantitative PCR (qPCR) assays were analyzed with one-way analysis of variance (ANOVA) followed by the Holm-Sidak all pairwise multiple comparison test when the distribution of delta-delta-Ct values passed the Shapiro-Wilk normality test.21 For the genes failed to pass the normality test (Agpt1, VEGF-A, TIMP4, PDGF-B, and Cox2), Kruskal-Wallis one-way ANOVA on ranks test was used followed by the Tukey all pairwise multiple comparison test.

### Results

Two closely-related CNS AVM mouse models were generated by conditionally deleting the Alk1 gene using Tg(SM22-Cre) deleter mice. In the first Alk1 (flox/flox);Tg(SM22-Cre) model, both copies of Alk1 were deleted by Cre recombinase expressed from SM22-Cre transgene (Flox-SM22Cre-del mice hereafter). In the second Alk1(flox/−);Tg(SM22-Cre) model, one of the two copies of Alk1 gene was constitutively deleted in all cells, mimicking human HHT, and the second Alk1 copy was deleted by SM22-Cre transgene (HHT+SM22Cre-del model hereafter). Of note, although SM22-Cre transgene was initially reported to induce Cre recombination in smooth muscle cells (SMCs) and cardiac muscles in embryos (Holtwick R, et al. Smooth muscleselective deletion of guanylyl cyclase-a prevents the acute but not chronic effects of anp on blood pressure. Proc Natl Acad Sci USA. 2002; 99:7142-7147), a wide-range and inconsistent Cre activation was observed in adults (data not shown). Furthermore, an involvement of both Alk1-null and Alk1-intact cells in AVM walls was identified (data not shown).

Both SM22Cre-del models caused partial lethality before 2 w of age (Tables 3 and 4). Intriguingly, the difference in the ratio of surviving pups at 2 w of age (Flox-SM22Cre-del pups: 47% vs HHT+SM22Cre-del: 15%, chi-square: p<0.01) was the only notable difference between the two SM22Cre-del models, and their gross phenotypes after 2 w of age were virtually indistinguishable. Therefore, the following analyses were performed mainly using the Flox-SM22Cre-del mice unless otherwise specified.

TABLE 3
Number of surviving pups per genotype at
2 weeks of age in Flox-SM22Cre-del model.

| Genotype | # of available pups at 2 weeks of age | | |
|---|---|---|---|
| Alk1(+/flox) | 96 | | |
| Alk1(flox/flox) | 88 | {close oversize brace} | (average 93.7) |
| Alk1(+/flox); Tg(SM22-Cre) | 97 | | |
| Alk1(flox/flox); Tg(SM22-Cre) (Flox-SM22Cre-del) | 44 | | |

Mating of Alk1(flox/flox) and Alk1(+/flox); Tg(SM22-Cre) mice was expected produce the listed four gentoypes at an equal ratio. Note that the number of available Flox-SM22Cre-del pups at 2 weeks of age was significantly lower than the average of the control genotypes (47% of the average, 44 vs. 93.7, chi-square: p < 0.01), indicating an early lethal phenotype

TABLE 4
Number of surviving pups per genotype in HHT+SM22Cre-deletion model.

| Genotype | # of available pups at newborn | | | at 2 weeks of age | | |
|---|---|---|---|---|---|---|
| Alk1(+/flox) | 49 | | | 159 | | |
| Alk1(flox/−) | 37 | {close oversize brace} | (average 37.7) | 183 | {close oversize brace} | (average 166.7) |
| Alk1(+/flox); Tg(SM22-Cre) | 27 | | | 158 | | |
| Alk1(flox/−); Tg(SM22-Cre) (HHT+SM22Cre-del) | 15 | | | 25 | | |

Mating of Alk1(flox/flox) and Alk1(+/−); Tg(SM22-Cre) mice was expected to produce the listed four genotypes at an equal ratio. Note that HHT+SM22Cre-del pups were available at a significantly lower rate at birth (39.8%, 15 vs 37.7, chi-square: p < 0.01) and at 2 w of age (15.0%, 25 vs 166.7, p < 0.01), indicating an early lethal phenotype.

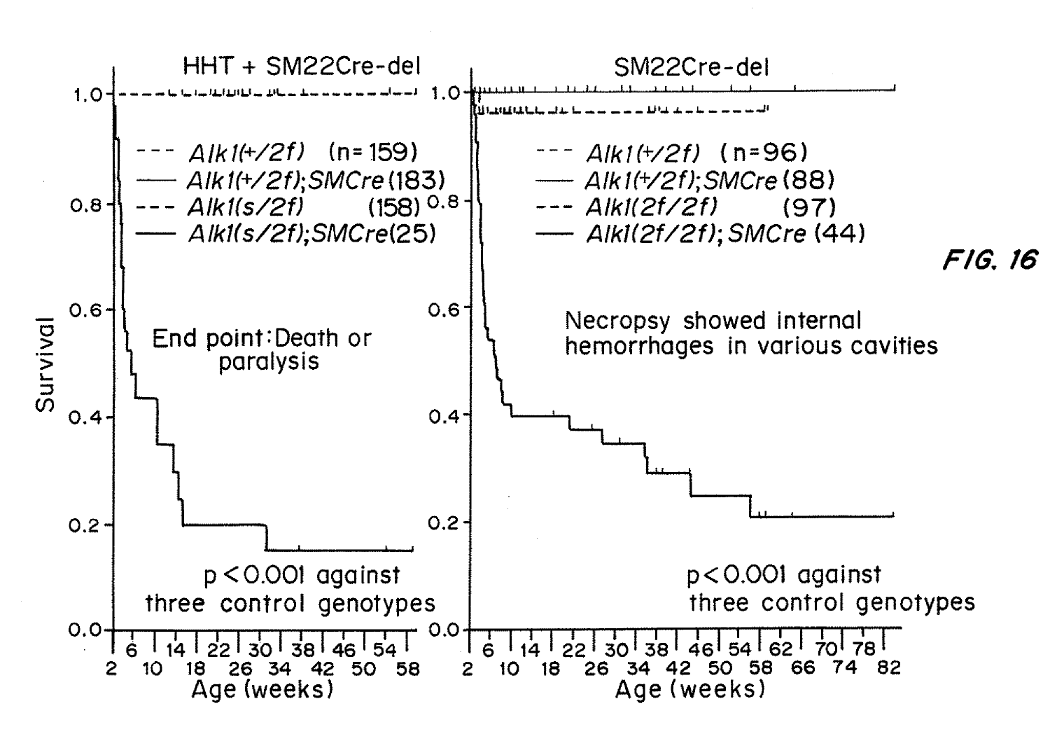

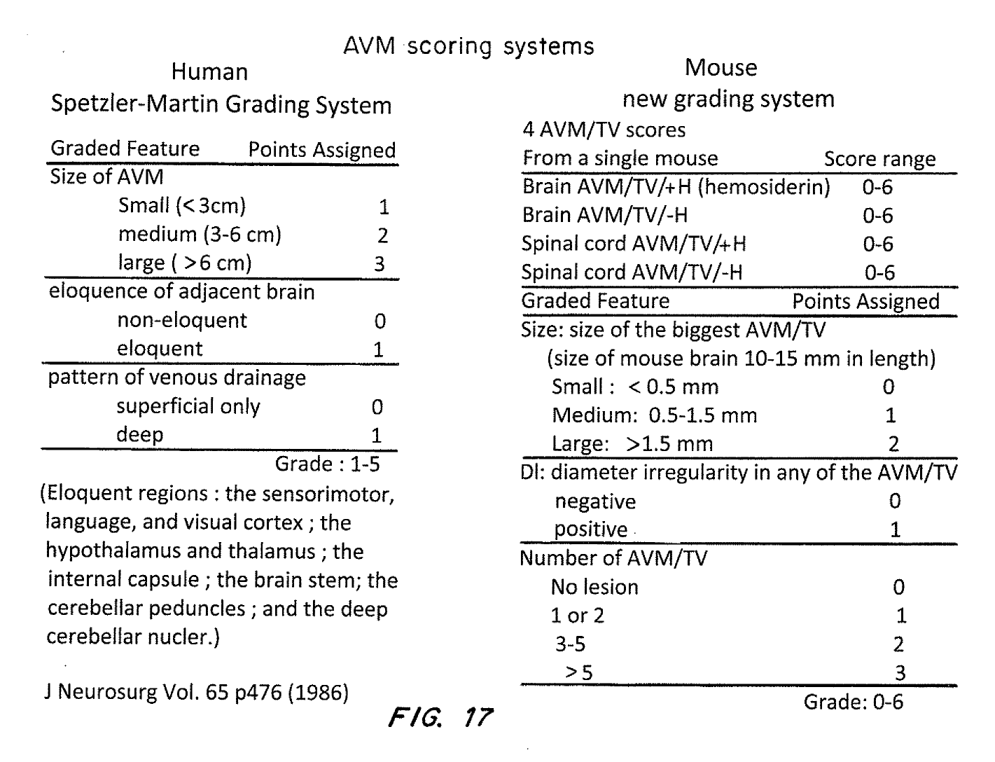

Survival curves of the two SM22Cre-del models after 2 w of age were essentially identical (log rank significance: p=0.31), and a majority of mice suffered from spontaneous death, hindlimb paralysis, or whole body paralysis during the following 8-13 weeks (FIG. 16). There was no significant difference between female and male survival curves in either model (both p>0.3, data not shown).

CNS hemorrhages were identified in the paralyzed SM22Cre-del mice. Hindlimb-paralyzed mice showed spinal cord hemorrhage in all cases, with AVM histology in some of them (data not shown). Severe intracranial hemorrhage was found in many of the completely paralyzed animals (data not shown). Since the blue latex solution injected through the cardiac left ventricle cannot enter microcapillaries due to its particle size (Park S O,et al. Real-time imaging of de novo arteriovenous malformation in a mouse model of hereditary hemorrhagic telangiectasia. J. Clin Invest. 2009; 119:3487-3496; Walker E J, et al. Arteriovenous malformation in the adult mouse brain resembling the human disease. Annals of neurology. 2011; 69:954-962) only arterial vessels should have been filled with latex (Holtwick R, et al. Smooth muscleselective deletion of guanylyl cyclase-a prevents the acute but not chronic effects of anp on blood pressure. Proc Natl Acad Sci USA. 2002; 99:7142-7147). However, cerebral veins of these animals were filled with blue latex, indicating a presence of AVMs. Overall, these findings indicate that Alk1-deletion by SM22-Cre transgene induced malformed vessels in the CNS, which caused internal hemorrhages and hence the early partial lethality.

Interestingly, the survival of the mice was considerably improved when the Flox-SM22Cre-del and HHT+SM22Cre-del mice surpassed 10 and 15 weeks of age, respectively (FIG. 16). The mice which survived this early lethal period (LP, ≦10 weeks of age) continued to show partial lethality, however, at much reduced rates during the stable period (SP, ≧16 weeks of age). In order to elucidate the cause of this change, two alternative hypotheses were considered. The first is that a small population of mice does not develop AVMs, and these non-AVM-bearing mice live much longer. The second is that most mice develop AVMs but the AVM walls stabilize once they reach SP age and become protected from lethal hemorrhages. The first hypothesis implies few or no AVMs in SP mice, whereas the second indicates presence of (multiple) AVMs in SP mice.

The cardiac blue latex injection was utilized to determine whether SP mice carry AVMs. All brain tissues in 15 SP mice (18-102 weeks of age) were found with various numbers, morphologies, and sizes of malformed vessels (data not shown). A number of malformed vessels showed direct connections between arteries and veins (AVMs), but there were also many tortuous vessels (TVs) that did not show an apparent connection to veins. In addition, many of the AVMs/TVs were accompanied by adjacent brown pigments, the hemosiderin deposits formed due to prior hemorrhages.

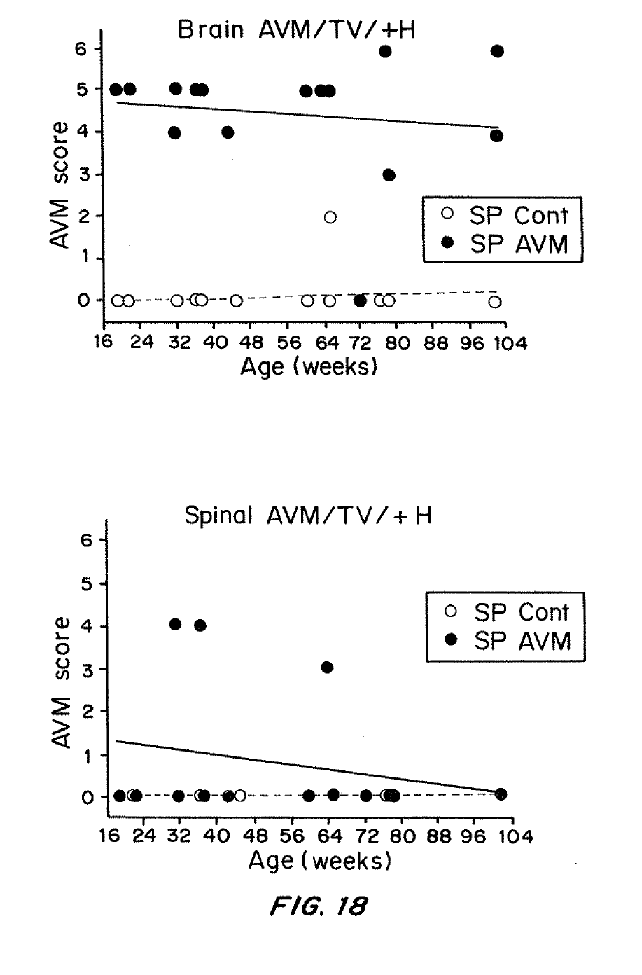

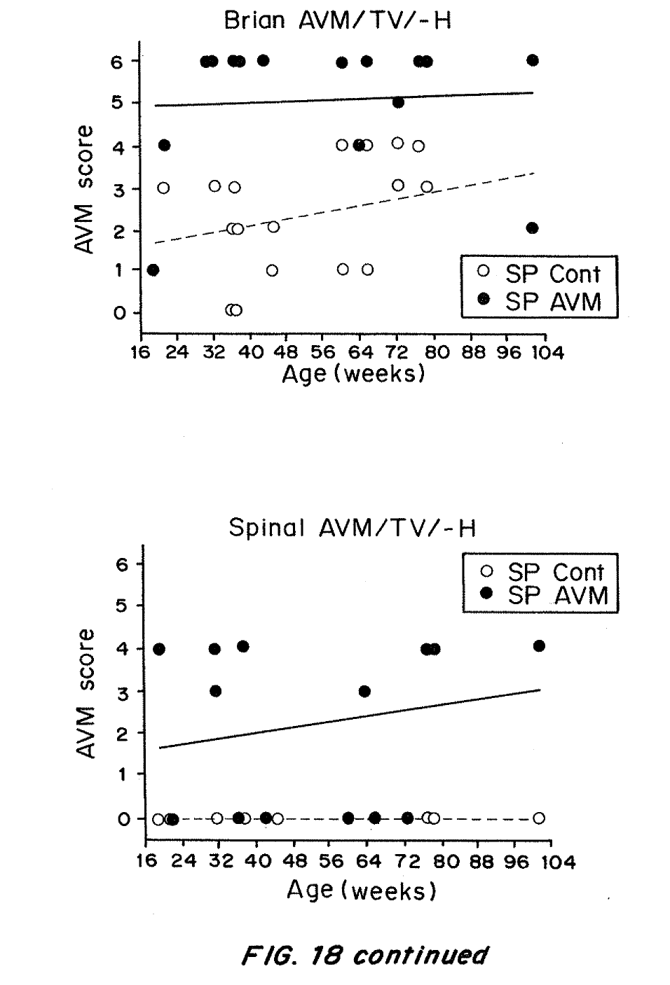

Intriguingly, sometimes relatively large AVMs were free of neighboring hemosiderin while some of smallest TVs were accompanied by hemosiderin (data not shown), indicating that any malformed vessels could cause hemorrhage. On a side note, it was found that malformed vessels in intestines of 4 SP animals (data not shown). In contrast, no AVMs were found in 22 littermate control mice except some TVs mostly in cerebellum region. These findings were confirmed by scoring the severities of AVMs/TVs using a newly-developed grading system (Table 1). The AVM/TV scores clearly showed that all SP SM22Cre-del mice carried a number of AVMs/TVs and most mice experienced hemorrhage (FIG. 18) (Soriano P. Generalized lacz expression with the rosa26 cre reporter strain. Nat Genet. 1999; 21:70-71).

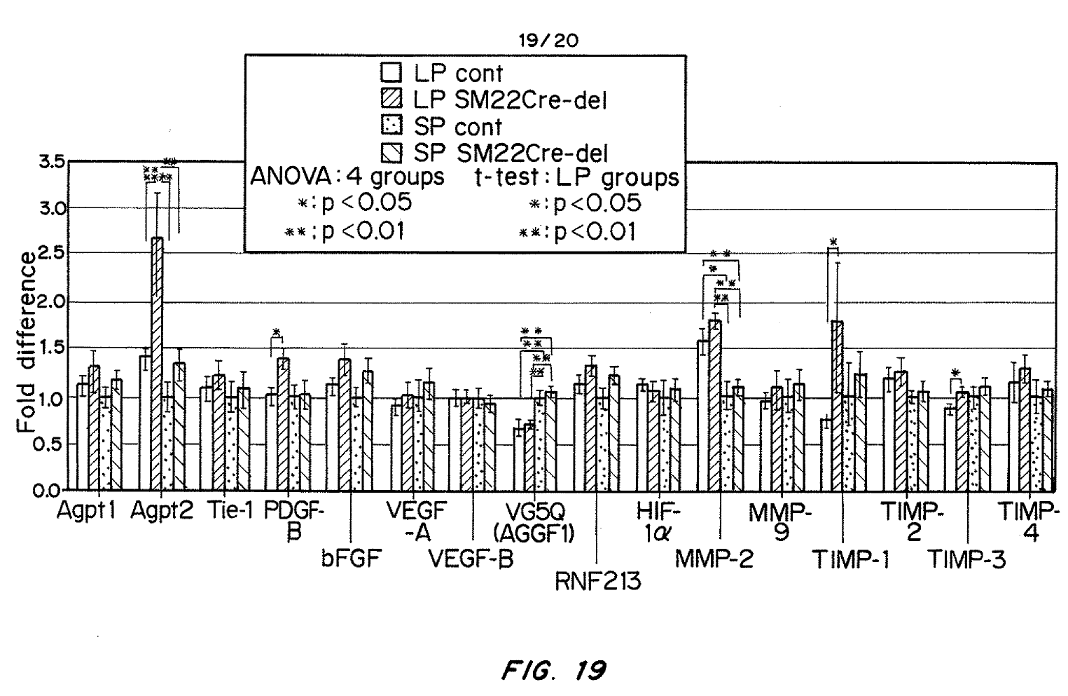

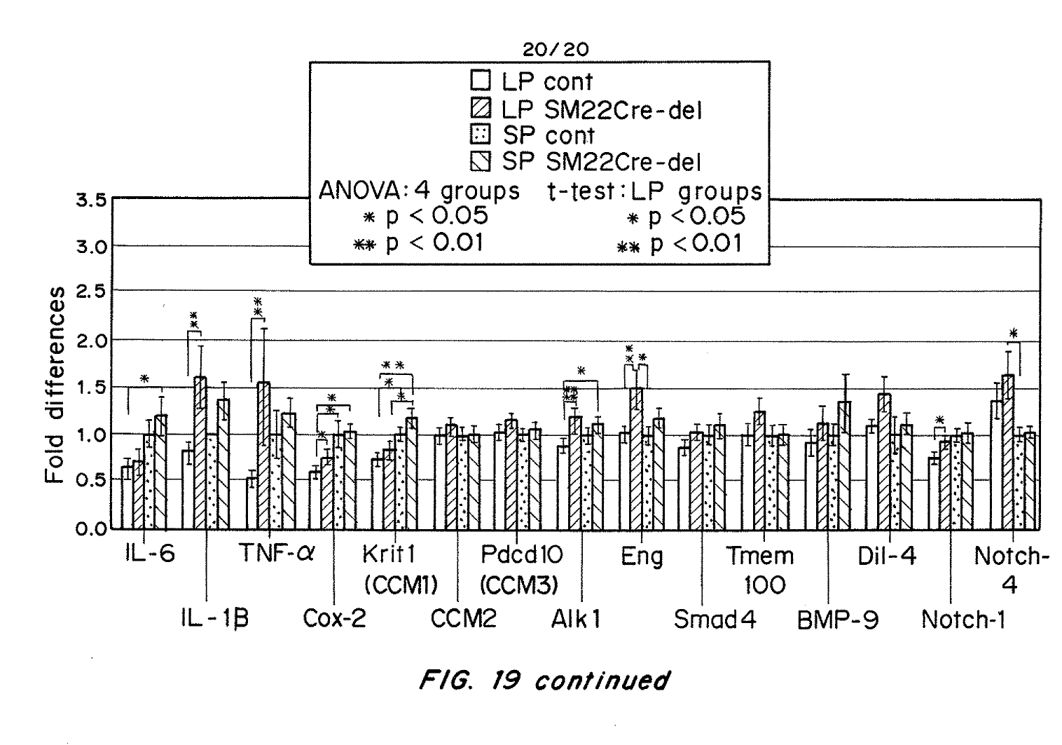

Since only the vasculature in LP SM22Cre-del mice was prone to bleed compared to the SP SM22Cre-del and LP/SP control mice, the LP SM22Cre-del brain may have a unique molecular environment that enhance the chances of hemorrhage from their vascular walls. Transcript levels of 31 candidate genes that may contribute to such environment were examined by qPCR using the brain tissues from these 4 groups, regardless of AVM presence (FIG. 19). Interestingly, Agpt2 was the only gene significantly upregulated in LP SM22Cre-del brains compared to the 3 other groups (all p<0.01). In addition, interleukin-1β (IL-1β) and tumor necrosis factor α were significantly upregulated in LP SM22Cre-del brains compared to LP control brains (p<0.05).

Of significance, proteins of some of these factors were induced in the AVM walls in a variegated fashion. Localizations of AVMs in LP brain (5-6 w of age) were identified by MR angiography, and their histological sections were prepared (data not shown). The nuclei of AVM wall cells were occasionally stained with cell proliferation markers (data not shown). As in other Alk1-deletion models (Park S O, et al. Alk5- and tgfbr2-independent role of alk1 in the pathogenesis of hereditary hemorrhagic telangiectasia type 2. Blood, 2008; 111:633-642; Park SO, et al. Real-time imaging of de novo arteriovenous malformation in a mouse model of hereditary hemorrhagic telangiectasia. J Clin Invest. 2009; 119:3487-3496; Walker E J, et al. Arteriovenous malformation in the adult mouse brain resembling the human disease. Annals of neurology. 2011; 69:954-962), SMC coverage of AVMs was inconsistent and variegated (data not shown). Similarly, Agpt2 and IL-1β expression was found in a variegated pattern in AVM walls. In addition, variegated expression of cyclooxygenase 2 (Cox2; PTGS2), a downstream molecule in inflammatory pathway, was also observed. Although SMC coverage and the expression of these proteins were both variegated, Agpt2 and Cox2 expression were actually often higher in the endothelial cell layer (data not shown). Interestingly, variegated focal recruitments of neutrophils and macrophages are reported in human AVM lesions (Chen Y, et al. Evidence of inflammatory cell involvement in brain arteriovenous malformations. Neurosurgery. 2008; 62:1340-1349; discussion 1349-1350).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. An activin receptor-like kinase 1 (Alk1) conditional knockout mouse with the phenotype Alk1(flox/flox)Tg(SM22-Cre).

2. The conditional knockout mouse of claim 1 further comprising transgenic expression of VEGF.

3. The conditional knockout mouse of claim 1, wherein the mouse comprises increased expression of an angiogenic or inflammatory factor selected from the group consisting of VEGF, Agpt2, Il-1β, TNF-α, Cox-2, and matrix metalloproteinase.

4. A method of treating or preventing arteriovenous vascular malformation (AVM) comprising administering an effective amount of a therapeutic composition, wherein the therapeutic composition reduces or inhibits an angiogenic or inflammatory factor selected from the group consisting of VEGF, Agpt2, Il-1β, TNF-α, Cox-2, and matrix metalloproteinase.

5. The method of claim 4, wherein the therapeutic composition comprises a Cox-2 inhibitor.

6. The method of claim 5, wherein the Cox-2 inhibitor is Celecoxib.

7. The method of claim 4, wherein the therapeutic composition comprises a matrix metalloproteinase inhibitor.

8. The method of claim 4, wherein a reduction or inhibition of Cox-2 results in a decrease or inhibition of inflammatory cytokines and chemokines.

9. A method of reducing or preventing hemorrhaging from AVM lesions comprising administering an effective amount of a therapeutic composition, wherein the therapeutic composition reduces or inhibits an angiogenic or inflammatory factor selected from the group consisting of VEGF, Agpt2, TNF-α, Cox-2, and matrix metalloproteinase.

10. A method for identifying molecular targets for therapeutic treatment of AVM, the method comprising identifying genes or proteins differentially expressed in a biological sample from the conditional knockout mouse of claim 1 compared to a control.

11. The method of claim 10 wherein the differential expression of genes is measured using quantitative polymerase chain reaction, microarray analysis, or northern blot analysis.

12. The method of claim 10 wherein the differential expression of proteins is measure using immunohistochemistry, western blot, mass spectroscopy, spectrophotometry, enzyme immuneassay, or a radioimmunoassay.

13. A method for determining the efficacy of a therapeutic treatment of AVM, the method comprising administering a candidate therapeutic to a first conditional knockout mouse of claim 1 and determining if the therapeutic prevents or reduces symptoms of AVM compared to a second conditional knockout mouse that is not administered the therapeutic.

14. The method of claim 13, wherein the symptom of AVM is the number or severity of AVM-associated hemorrhages.

15. The method of claim 13, wherein the symptom of AVM is increased expression of angiogenic or inflammatory factors.

16. The method of claim 15, wherein the angiogenic or inflammatory factor is selected from the group consisting of VEGF, Agpt2, Il-1β, TNF-α, Cox-2, or a matrix metalloproteinase.
